(19) United States
(12) Patent Application Publication
(10) Pub. No.: US 2011/0243854 A1
(43) Pub. Date: Oct. 6, 2011

(54) Methods and systems for identifying modulators of longevity

(75) Inventors: Douglas C. Wallace, Irvine, CA (US); James Jiayuan Tong, Tustin, CA (US)

(73) Assignee: The Regents of the University of California, Oakland, CA (US)

(21) Appl. No.: 12/925,539

(22) Filed: Oct. 20, 2010

Related U.S. Application Data
(63) Continuation of application No. 11/901,249, filed on Sep. 14, 2007, now abandoned.
(60) Provisional application No. 60/930,421, filed on May 15, 2007.

(51) Int. Cl. A61K 49/00; G01N 33/48; C40B 60/08; C12Q 1/02; A61K 31/7076; A61K 31/135; A61K 31/409; A61K 31/555; A61K 38/02; A61K 48/00; C12Q 1/48; C12Q 1/44; C12Q 1/26; C12Q 1/28; A61P 35/00; A61P 43/00

(52) U.S. Cl. 424/9.2; 800/3; 506/37; 435/29; 514/47; 514/641; 514/410; 514/185; 514/1.1; 514/44 R; 514/44 A; 435/15; 435/19; 435/25; 435/28; 514/19.3

(57) ABSTRACT

Methods of treating disorders such as neurofibromatosis-1 are provided, including methods in which catalytic antioxidants such as metalloporphyrins are administered. Methods of regulating longevity, and methods and systems for screening for modulators of aging or longevity, are also provided. In addition, related transgenic animals are described.

### CROSS-REFERENCE TO RELATED APPLICATIONS

This application is a non-provisional utility patent application claiming priority to and benefit of the following prior provisional patent application Ser. No. 60/930,421, filed May 15, 2007, entitled “NF1 GENE ASSAYS AND APPLICATIONS” by Douglas L. Wallace and James Jiayuan Tong, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant Nos. AR-47752, AG13154, AG24373, AG01751, DK73691, NS21328 and NS41850 from the National Institutes of Health. The government may have certain rights to this invention.

### FIELD OF THE INVENTION

The field of the invention relates to methods of treating NF1 disorders, methods of regulating longevity, and methods and systems for screening for modulators of aging or longevity. Related transgenic cells, animals and systems are also described.

### BACKGROUND OF THE INVENTION

Neurofibromatosis-1 (NF1) is one of the most common neurogenetic diseases, with a prevalence of 1 in 3,000 worldwide¹⁻³. More than 10% of humans harboring mutations in the NF1 gene develop malignant tumors early in life¹⁻², and the life expectancy of individuals with NF1 is reduced by at least 10 to 15 years¹⁻³. In addition to café-au-lait spots and neurofibromas, NF1 manifestations include learning disabilities and developmental abnormalities (e.g., refs. 4-5).

There is need for elucidation of the molecular links between NF1 gene mutations and the pathophysiology of NF1 disorders. Further, there is need for effective therapies for NF1.

There is also need for therapies to increase life span and decrease aging, particularly for humans, regardless of the presence of NF1 mutations. Studies in model organisms have shown that the aging process is regulated by a conserved mechanism, and life span extension has been achieved in multiple animal systems by inactivation of the insulin-like receptor signal transduction pathway (Kenyon (2001) “A conserved regulatory system for aging” Cell 105:165-8). When activated by insulin-like ligands, this pathway activates the Akt kinase that phosphorylates and inactivates the forkhead transcription factors. Active forkhead transcription factors upregulate MnSOD and the peroxisome proliferator-activated receptor γcoactivator (PGC-1α) gene. The PGC-1α protein, in turn, interacts with multiple transcription factors to upregulate mitochondrial biogenesis. Inactivation of the insulin-like growth factor receptor pathway is therefore predicted to upregulate mitochondrial biogenesis and reduce production of mitochondrial reactive oxygen species that may contribute to senescence.

Although considerable progress has been made in understanding aging, there is still need for elucidation of other pathways that influence aging, as well as for novel ways to increase longevity and decrease the effects of aging.

Among other benefits, the present invention meets the above needs by providing the identity of a key pathway that mediates the effects of NF1 and that ties NF1 activity to aging and longevity, by providing methods for treating NF1 disorders, by providing methods for screening for modulators of aging and longevity, and by providing methods for regulating longevity. A complete understanding of the invention will be obtained upon review of the following.

### SUMMARY OF THE INVENTION

A key pathway that ties NF1 activity to aging and longevity has been identified, leading to various aspects of the present invention as set forth below, including, without limitation, methods and systems for screening for modulators of aging or longevity, methods for regulating longevity, and methods for treating NF1 disorders.

One aspect of the present invention includes methods of screening for a modulator of aging or longevity. In the methods, a non-human animal with an artificial mutation in, or an artificial disruption of expression of, a gene that encodes a component of or that regulates an adenylyl cyclase/cyclic AMP/protein kinase A pathway in the animal, wherein the mutation or disruption is correlated with an aging or longevity trait for the non-human animal, is provided. The modulator is administered to the non-human animal. An effect of the modulator on a phenotype of the non-human animal, wherein the phenotype is correlated to said mutation or disruption, is monitored.

Optionally, the mutation is in a gene such as a neurofibromatosis-1 gene, an adenylyl cyclase gene, a cAMP phosphodiesterase gene, or a protein kinase A gene. For example, the mutation can result in inactivation or overexpression of the neurofibromatosis-1 gene.

Suitable non-human animals that can easily be screened for modulators include, but are not limited to, insects, including a Drosophila such as a Drosophila melanogaster, nematodes such as Caenorhabditis elegans, and rodents. The modulator is optionally administered by feeding it to the non-human animal.

Any of a variety of different assays can be employed, depending on the phenotype of interest. For example, in one class of embodiments, the phenotype is a life span phenotype, and monitoring the effect of the modulator comprises performing a longevity assay that measures the life span of the animal in presence of the modulator. In another exemplary class of embodiments, the phenotype is a stress resistance phenotype, and monitoring the effect of the modulator involves performing a stress resistance assay that measures stress resistance of the animal in presence of the modulator. In this class of embodiments, the stress resistance phenotype optionally comprises reduced resistance to heat or oxidative stress as compared to an isogenic or near isogenic animal that lacks the mutation or disruption, and monitoring the effect of the modulator comprises detecting increased resistance to heat or oxidative stress caused by the modulator.

In another exemplary class of embodiments, the phenotype comprises a physical activity or locomotion phenotype, and monitoring the effect of the modulator comprises performing a physical activity assay that measures physical activity of the animal in presence of the modulator. For example, the animal can be an insect, and the physical activity assay can include measuring up climbing/escape response activity of the insect.

In yet another exemplary class of embodiments, the phenotype comprises an alteration in mitochondrial respiration, and monitoring the effect of the modulator comprises performing a mitochondrial respiration activity assay that measures mitochondrial respiration in cells or tissues of the animal, or in an extract thereof, in presence of the modulator. In a related class of embodiments, the phenotype comprises a mitochondrial respiration trait and monitoring the effect of the modulator comprises performing a mitochondrial respiration activity assay that measures mitochondrial respiration in the animal, in cells or tissues of the animal, or in an extract thereof, after administration of the modulator.

Other exemplary phenotypes that can be monitored, e.g., as described herein, include (a.) cAMP concentration in the animal, in cells or tissues of the animal, or in an extract thereof, (b.) complex I activity in the animal, in cells or tissues of the animal, or in an extract thereof, (c.) citrate synthase activity in the animal, in cells or tissues of the animal, or in an extract thereof, (d.) mitochondrial ROS production in the animal, in cells or tissues of the animal, or in an extract thereof, (e.) mitochondrial respiratory control ratio (state III O₂ consumption rate/state IV O₂ consumption rate) in the animal, in cells or tissues of the animal, or in an extract thereof, (f.) ATP production rate when metabolizing NADH-linked substrates in the animal, in cells or tissues of the animal, or in an extract thereof, (g.) aconitase activity in the animal, in cells or tissues of the animal, or in an extract thereof, (h.) superoxide dismutase or catalase activity in the animal, in cells or tissues of the animal, or in an extract thereof, and (i.) reproductive capacity of the animal.

Optionally, in any of the above embodiments, the phenotype of the animal in the presence of the modulator is compared, as a control, to that of an isogenic or nearly isogenic animal in the absence of the modulator.

Exemplary modulators include, but are not limited to, a cAMP analog, an antioxidant, a catalytic antioxidant, a metalloporphyrin catalytic antioxidant, or the like.

Another general class of embodiments provides methods of screening for a modulator that increases life span. The methods include the steps of administering a putative modulator to a non-human animal (e.g., an insect), and testing for increased neurofibromin expression or activity in the animal following administration of the modulator, wherein increased neurofibromin expression or activity in the animal correlates with increased life span.

Yet another general class of embodiments provides methods of screening for a modulator that increases life span. In this class of embodiments, the methods include administering the modulator to a non-human animal (e.g., an insect, nematode, or rodent) and testing for changes in adenylyl cyclase/cyclic AMP/protein kinase A pathway component expression, activity, or concentration.

Systems for screening for modulators of aging or longevity are also a feature of the invention. For example, one class of embodiments provides a system for screening for a modulator compound that modulates an aging related behavioral phenotype. The system comprises an array of non-human animals in containers, a behavior monitoring module that monitors the behavioral phenotype of the animals in the containers in the presence of the modulator, and a correlation module that correlates behavior of the animal to aging or life span. In one aspect, the behavior monitoring module monitors physical activity of the animals, e.g., climbing/escape response behavior.

Essentially all of the features noted for the methods above apply to this class of embodiments as well, as relevant. It is worth noting that the animals optionally comprise a mutation or disruption in a gene that encodes a component of or that regulates an adenylyl cyclase/cyclic AMP/protein kinase A pathway in the animal, e.g., in a gene selected from the group consisting of a neurofibromatosis-1 gene, an adenylyl cyclase gene, a cAMP phosphodiesterase gene, and a protein kinase A gene. For example, the mutation can result in inactivation or overexpression of the neurofibromatosis-1 gene. Exemplary animals include, but are not limited to, rodents, nematodes, and insects, including Drosophila such as Drosophila melanogaster.

A related class of embodiments provides a system for screening for modulator compounds that modulate an aging related behavioral trait. The system includes, e.g., an array of insects in containers and a behavior monitoring module that monitors physical activity of the insects in the array following administration of the modulator compounds. In one embodiment, the system comprises an automated shaker or tapper that shakes or taps the containers of the array.

Again, essentially all of the features noted above apply to this class of embodiments as well, as relevant. For example, the physical activity monitored can be an up climbing/escape response behavior. The insects optionally comprise a mutation or disruption in a gene that encodes a component of or that regulates an adenylyl cyclase/cyclic AMP/protein kinase A pathway in the insect, e.g., in a gene selected from the group consisting of a neurofibromatosis-1 gene, an adenylyl cyclase gene, a cAMP phosphodiesterase gene, and a protein kinase A gene. For example, the mutation can result in inactivation or overexpression of the neurofibromatosis-1 gene. In one class of embodiments, the insects are Drosophila melanogaster.

Another general class of embodiments provides methods for regulating longevity of an animal. In these embodiments, an adenylyl cyclase/cyclic AMP/protein kinase A pathway in the animal is modulated. A related general class of embodiments provides methods of regulating longevity of an animal; in these embodiments, neurofibromin expression or activity in the animal is modulated. In either class of embodiments, the animal optionally comprises a mutation in one or more of a neurofibromatosis-1 gene, an adenylyl cyclase gene, a cAMP phosphodiesterase gene, and a protein kinase A gene. The methods optionally include increasing neurofibromin expression or activity in the animal. The methods can include administering a longevity modulator to the animal over an extended period of time.

Transgenic animals related to or of use in the methods and systems of the invention are also featured. Accordingly, one general class of embodiments provides a transgenic non-human animal comprising a knock out or knock down mutation in one or more copies of an NF1, an adenylyl cyclase, a cAMP phosphodiesterase, or a PKA gene in the genome of the animal, wherein the animal further comprises a recombinant NF1, adenylyl cyclase, cAMP phosphodiesterase, or PKA gene.

In one class of embodiments, the animal is an insect, the gene is an NF1 gene, and the recombinant NF1 gene is under the control of a heterologous inducible promoter. Similarly, in one class of embodiments, the animal is an insect, the gene is an adenylyl cyclase gene, and the recombinant adenylyl cyclase gene is under the control of a heterologous inducible promoter. In one class of embodiments, the animal is an insect, the gene is a PKA gene, and the recombinant PKA gene is under the control of a heterologous inducible promoter. The inducible promoter can be, e.g., a heat shock promoter.

Modulators identified by the methods or systems of the invention are also a feature of the invention. A modulator identified using a method or system herein is optionally administered to an animal, including a human patient, to modulate (e.g., extend) longevity or life span, to treat NF1, for testing in cell or animal-based anti-cancer models, to use in the treatment of cancer, to treat an age-related metabolic or degenerative disease, to increase activity and muscle strength, or the like.

In one aspect, the invention includes methods of treating an NF1 disorder. In the methods, a catalytic antioxidant is administered to a patient suffering from the disorder. Optionally, the catalytic antioxidant to be administered is a metalloporphyrin.

### BRIEF DESCRIPTION OF THE DRAWINGS

FIG. 1 Panels A-E illustrate that NF1 deficiency shortens D. melanogaster life span through adenylyl cyclase/cAMP/PKA signaling. Panel A presents a graph of percentage survival over time for various strains, demonstrating that inactivation of the NF1 gene reduced life spans in females. NF1 mutant strains (NF1^{P1}/and NF1^{P2}) and their intercross (NF1^{P1/P2}) showed shorter life spans than the control K33 (log-rank test comparing NF1^{P1} and NF1^{P2} with control K33, P<0.0001). Expression of D. melanogaster NF1 transgene in hsNF1/+; NF1^{P2} rescued the NF1 aging phenotype (log-rank test: hsNF1/+; NF1^{P2} versus NF1^{P1} or NF1^{P2}, P<0.001; hsNF1/+; NF1^{P2} versus K33, P=0.28) (K33, n=260; NF1^{P1}, n=260; NF1^{P2}, n=260; NF1^{P1/P2}, n=260; hsNF1/+; NF1^{P2}, n=280, distributed in eight tubes). Panel B presents a graph of percentage survival over time for various strains, demonstrating that inactivation of the NF1 gene reduced life spans in females and that neurofibromin modulates life span through the adenylyl cyclase/cAMP/PKA pathway, as illustrated by median and maximal life spans (mean±s.d.). rutabaga mutants, including rut¹ and rut²⁰⁸⁰, had shorter life spans. NF1 mutation did not further reduce the life span on the rut background, rut; NF1^{P2} (log-rank test, P=0.127). Expression of PKA in hsPKA/+; NF1^{P2} flies rescued the NF1 aging phenotype (NF1^{P2}, n=360; rut¹, n=240; rut²⁰⁸⁰, n=360; rut; NF1^{P2}, n=260; dnc; NF1^{P2}, n=260, hsPKA/+; NF1^{P2}, n=320, hsRas^{V12}/+; K33, n=320, hsRaf*^{M7}/+; K33, n=360, in eight repeats). Panel C presents a table of median and mean life spans for males and females of various strains. Panel D presents a graph of locomotive index over time, illustrating NF1/adenylyl cyclase/PKA regulation of up-climb behavior under heat stress (20 min, 37° C.). Locomotive index (LI)=startle-induced up-climbing. Time (τ; in min) to recovery of LI after heat shock. There was a significantly prolonged recovery time (τ) in rut mutant flies (τ=92±4) compared with control flies (τ=27±3, P<0.005, t test). Panel E presents a bar graph showing recovery time (τ) for various strains, illustrating NF1-regulated heat stress recovery via the adenylyl cyclase/cAMP/PKA pathway (mean±s.d. of six experiments; total of 100-140 flies per genotype). NF1^{P1}, NF1^{P2}, NF1^{P1/P2} and rut; NF1^{P2} showed delayed recovery time. This phenotype was rescued by (i) expressing neurofibromin in hsNF1/+; NF1^{P2}, (ii) elevating cAMP by dunce, dnc; NF1^{P2} and (iii) overexpressing PKA in hsPKA/+; NF1^{P2} flies (*, P<0.005, t test). hsRas^{V12}/+; K33 and hsRaf*^{M7}/+; K33 showed a similar τ as control K33 flies.

FIG. 2 Panels A-G illustrate that mitochondrial energy deficiency and increased oxidative stress of NF1 mutants can be rescued by antioxidants. Panel A presents a bar graph showing increased oxidative stress indicated by heightened paraquat sensitivity, reduced aconitase activity and reduced mitochondrial ATP production in K33, NF1^{P2}, rut, rut; NF1^{P2}, hsNF1/+; NF1^{P2}, hsPKA/+; NF1^{P2} and hsNF1/+; K33 flies. Panel B shows a bar graph illustrating that reactivation of aconitase activity by dithiothreitol and iron confirms activity loss by oxidative stress. For Panels A and B: Paraquat (20 mM), six experiments (180-220 females/genotype). Aconitase activities, mean±s.d., five experiments, 400 flies/genotype. ATP synthesis calculated from mitochondrial respiration data (FIG. 9), mean±s.d., five experiments, 75 males and females flies per experiment. *, P<0.05; **, P<0.01, t test (compared with control K33). Panel C presents a graph of superoxide signal, evaluated by MitoSOX fluorescence when metabolizing pyruvate and malate (arrow), for control K33 and NF1^{P1/P2} mitochondria. Panel D presents a bar graph showing that superoxide production rates, measured from the initial slope of MitoSOX fluorescence, increased immediately after substrate addition (mean±s.d., six experiments; 50 male and 50 female flies/experiment; 600 flies/genotype). The superoxide production increased significantly (by 101% in NF1^{P2} and 103% in rut and rut; NF1^{P2} mutants) but was restored in hsNF1/+; NF1^{P2} flies (*, P<0.05; mean±s.d. of six experiments; 50 males and 50 females/experiment; 600 flies/genotype). Panels E and F present graphs showing that MnTBAP (10 μM) and MnTDEIP (10 μM) feeding extended NF1^{P1/P2} life spans (eight experiments; 320-400 flies/group; log-rank test, P<0.0001 for both treatments) for both males (Panel E) and females (Panel F). There were no significant weight changes throughout the experiment. Panel G presents a bar graph showing that MnTBAP and MnTDEIP feeding rescued delayed heat stress recovery time (*, P<0.005, t test).

FIG. 3 Panels A-F illustrate life extension in flies by overexpression of NF1. Panels A and B show hsNF1/+; K33 versus K33 flies at 25° C. (log-rank test: P<0.0001 for males and females; seven or eight experiments with 40 flies each; total of 280 or 320 flies per category); males are shown in Panel A and females in Panel B. Panels C and D show age-specific mortality rates of hsNF1/+; K33 and K33 flies (males in Panel C and females in Panel D) plotted on a natural log scale versus time, fit by Gompertz model (ln(μₓ)=ln(μ₀)+a^{x}), where μₓ=mortality rate at age x, μ₀=baseline mortality (intercept as ln(μ₀)) and a=change of mortality with age (slope of the trajectory). Values are given in Table 2. In panels A-D, NF1 overexpression was achieved using the heat shock promoter. Panels E-F present graphs of percentage survival over time for males (Panel E) and females (Panel F). Offspring from the crosses between homozygous Armadillo-GAL4 and two independent homozygous UAS-D. melanogaster NF1 transgenic lines yield double heterozygous flies (Arm-GAL4/UAS-dNF1), compared with individual transgene heterozygotes (Arm-GAL4/+ and UAS-dNF1/+). Results of log-rank test are shown (P<0.0001 for both males and females, six experiments with 40 flies per experiment, total of 240 flies per category). In panels E-F, NF1 overexpression was achieved using the GAL4-UAS system.

FIG. 4 Panels A-H illustrate phenotypic analysis of hsNF1/+; K33 versus K33 flies. Panel A presents a bar graph showing enhanced reproductive fitness of hsNF1/+; K33 flies (mean fertility±s.d. of daily offspring from five pairs of male and female flies per genotype, 12 replicates; P<0.05 every day; t test). Panel B presents a graph showing that the body weight of hsNF1/+; K33 males was similar to that of K33 males, but the weight of hsNF1/+; K33 females was higher than that of K33 females (mean weight per fly±s.d., four replicates at each time point). Panel C presents a bar graph showing similar body lengths of hsNF1/+; K33 and K33 flies at 15 d (mean length per fly±s.d. from 13 replicates; P=0.16 between males and P=0.062 between females for hsNF1/+; K33 versus K33 flies; t test). Panel D presents a graph illustrating resistance to heat shock of hsNF1/+; K33 flies in locomotive test, measured by up-climbing ability before, during and after a 20-min exposure to 37° C. (20 male and 20 female flies in each tube; for hsNF1/+; K33, n=7, total of 280 flies; for K33, n=5, total of 200 flies). Panels E-F present bar graphs of mean survival time for males (Panel E) and females (Panel F), showing enhanced resistance to paraquat oxidative stress of hsNF1/+; K33 flies. Males lived 51% longer (P=0.012, t=2.23, df=10, t test), and females lived 56% longer (P=0.007, t=2.25, df=10, t test) than K33 flies (40 flies per tube, n=6, total of 240 flies per category). Panels G-H present graphs of percentage survival over time for males (Panel G) and females (Panel H), showing that hsNF1/+; K33 and K33 flies had a similar desiccation tolerance (Wilcoxon test, 40 flies per tube, n=6, total of 240 flies per category).

FIG. 5 Panels A-H illustrate that upregulation of cAMP/PKA signaling extends D. melanogaster life span. Panels A-D present graphs of percentage survival over time for males fed dibutyryl-cAMP (Panel A), females fed dibutyryl-cAMP (Panel B), males fed 8-bromo-cAMP (Panel C), and females fed 8-bromo-cAMP (Panel D), showing that feeding dibutyryl-cAMP and 8-bromo-cAMP extends life spans of male and female w¹¹¹⁸ flies (five experiments, with a total of 180-200 flies; log-rank test, P<0.001 for control flies versus those treated with 1 μM and 10 μM of either compound). Panels E-F present graphs of percentage survival over time for males (Panel E) and females (Panel F), showing that a cAMP phosphodiesterase mutant (dunce) extends life span on the Canton-S (CS) background (log-rank test, P<0.0001 for males and females, five experiments, 220-240 flies per category). Panels G-H present graphs of percentage survival over time for males (Panel G) and females (Panel H), showing that expression of a constitutive protein kinase A (PICA) catalytic subunit (hsPKA*) extends life span in hsPKA*/+; K33 flies compared with control K33 flies (log-rank test, P<0.0001 in males and females, six experiments for K33 and eight experiments for hsPKA*/+; K33 flies, with 40-50 flies per experiment, ≧240 flies per category).

FIG. 6 Panels A-I illustrate that NF1 overexpression increases complex I respiration and activity, reduces ROS production and protects aconitase activity. Respiratory rate=atomic oxygen consumed/min/mg mitochondrial protein. Panel A presents a bar graph illustrating that mitochondrial respiration with pyruvate+malate with ADP (state III) is elevated in hsNF1/+; K33 flies versus K33 flies (*, P=0.02, t test, left column) but not without ADP (state IV) (P=0.84, right column). Panel B presents a bar graph illustrating that NF1 expression did not alter P/O ratio (P=0.69). Panel C presents a bar graph illustrating that rotenone-sensitive complex I activity (NADH:DB coenzyme Q analog oxidoreductase assay) is elevated in hsNF1/+; K33 flies (mean±s.d., six experiments; 50 males and 50 females/experiment; *, P=0.04, t test). No difference in residual activity was found in the presence of rotenone (4 μM) for hsNF1/+; K33 (residual activity=6.4±1.9) versus K33 (residual activity=5.7±2.7) flies (P=0.79, t test). Panel D presents a bar graph illustrating that mitochondrial respiration using succinate with ADP (state III, left column) or without ADP (state IV; right column) is similar between hsNF1/+; K33 and K33 flies (P=0.48 for III and P=0.33 for IV). Panel E presents a bar graph illustrating that their P/O ratios are similar (P=0.21) (mean±s.d. of six experiments (Panels A-B) or four experiments (Panels D-E), 50 males+50 females/experiment). Panel F presents a bar graph illustrating that the activity of mitochondrial TCA cycle enzyme citrate synthase is the same in both genotypes (P=0.64, t test; three experiments). Panel G presents a bar graph illustrating that H₂O₂ secretion is reduced by 58% in hsNF1/+; K33 mitochondria versus K33 mitochondria (**, P<0.01, t test; three experiments). Panel H presents a graph illustrating that mitochondrial aconitase activity is higher in hsNF1/+; K33 versus K33 flies throughout life (*, P<0.05; **, P<0.01, t test; three experiments, 50 males and 50 females per experiment). Panel I presents a bar graph illustrating that the difference in mitochondrial aconitase activity of 15-d-old K33 and hsNF1/+; K33 flies was reactivated to a similar level with dithiothreitol and iron (**, P<0.01, t test; six experiments, 50 flies of mixed gender per experiment; 300 flies for each genotype).

FIG. 7 schematically illustrates the mechanism of neurofibromin-regulated life span. Neurofibromin regulates adenylyl cyclase (AC), which converts ATP to cAMP, activating PKA. PKA then increases mitochondrial respiration, possibly by phosphorylating complex I subunits, leading to an increase in ATP synthesis and a reduction in mitochondrial ROS production. By increasing ATP production and inhibiting ROS production, neurofibromin promotes longevity. The genetic and pharmacological manipulations discussed in the text influence the process (arrows) in a positive or negative manner. cAMP might modulate mitochondrial energy production and ROS generation (and thus life span) by regulating nuclear gene transcription as well. Catalytic antioxidants MnTBAP and MnTDEIP increase life spans in NF1 mutants by inhibiting a negative factor, ROS. Although D. melanogaster data do not support the role of Ras in neurofibromin-regulated longevity, possible connections in mammals are indicated by dashed lines. PDE, phosphodiesterase.

FIG. 8 Panels A-B presents bar graphs illustrating normal desiccation tolerance in NF1 mutants (females in Panel A and males in Panel B). 40 flies per tube, n=6, mean±SD, total of 240 flies per category.

FIG. 9 presents a bar graph illustrating that NF1/AC/cAMP signaling modulated mitochondrial respiration. NF1 or rut mutants reduced NADH-linked and ADP-stimulated (state III) respiration without altering ADP-independent (state IV) respiration. Expression of one copy of heat shock controlled Drosophila NF1 gene in hsNF1/+; NF1^{P2} restored normal state III rate in NF1^{P2} flies (*: P<0.05, t-test). NADH-linked respiration is driven by pyruvate and malate which reduce NAD⁺ to NADH as they are metabolized in the mitochondria. NADH is reoxidized back to NAD⁺ by OXPHOS complex I, n=4, mean±SD, total 150 flies per experiment.

FIG. 10 Panels A-B demonstrate intact oxidative stress defense enzymes in NF1 mutants. Panel A presents a bar graph of SOD activity. Panel B presents a bar graph of total catalase activity. Total and mitochondrial superoxide dismutase (MnSOD) and catalase activities were similar between NF1 mutant and control mitochondria, mean±SD of 4 experiments, 400 flies per genotype (P=0.32 for total SOD; P=0.98 for MnSOD, t−test; P=0.64 for catalase).

FIG. 11 Panels A-C illustrate that life extension was correlated with NF1 expression level. Panel A presents a western blot showing that NF1 expression is increased in hsNF1/+; K33 flies at 25° C. Western blots were used to detect the 280 kD NF1 protein. The NF1 level of hsNF1/+, K33 was greater than in K33, while NF1 level of K33 was greater than the null mutant NF1^{P1}. Panel B presents a bar graph showing that similar increases in NF1 protein levels were generated by both the heat shock and GAL4-UAS systems, detected by western blot measured using Image J densitometry analysis, mean±SD of 3 experiments (*: P<0.05, t−test). Panel C presents a graph showing survivalship of hsNF1/+; K33 and control K33 flies at 18° C. Each data point is the mean±SD of 13 experiments with 40 flies per experiment, 520 flies total per category. Both genotypes were maintained at 25° C. throughout embryogenesis and 4 days into adulthood before transfer to 18° C. Flies were briefly exposed to room temperature (24° C. to 25° C.) for 15 minutes during food replacements every 4 days. No difference was observed in maximal life spans (P=0.783, t-test), though minimal differences were seen in mean life spans (P=0.024, Logrank test). NF1 levels in these flies were shown.

FIG. 12 Panels A-B illustrate life extension in Drosophila by neuronal over-expression of NF1 via combining the neuronal promoter ELAV-GAL4 line with the independent UAS-dNF1 transgenic lines (ELAV-GAL4/UAS-dNF1) versus the individual transgene heterozygotes (ELAV-GAL4/+ or UAS-dNF1/+). Logrank test: P<0.0001 for both males (Panel A) and females (Panel B), 6 experiments, 40 flies per experiment, 240 flies per category.

FIG. 13 Panels A-B illustrate that elevated cAMP in NF1 over-expression flies and cAMP feeding did not alter fly body weights. Panel A presents a bar graph showing that NF1 over-expressing flies showed higher cAMP concentrations than controls (*: P<0.02), from 4 experiments of 25 male and 25 female flies per experiment, total of 200 flies per genotype. Panel B presents a bar graph showing that dietary supplementation of cAMP did not affect body weight. Male and female w¹¹¹⁸ flies were fed regular food containing 0 μM, 1 μM and 10 μM dibutyryl—cAMP until 45 days of age. Groups of 10 flies per treatment were weighted. n=4, average weight per fly±SD.

FIG. 14 Panels A-B illustrate that NF1 over-expression did not influence the level of whole fly dephosphorylated forkhead transcription factor (dFOXO) levels or a total superoxide dismutase (SOD) or MnSOD activities. Panel A illustrates western blot analysis of FOXO1 detected by an antibody (Cell Signaling) that preferably binds to dephosphorylated FOXO. Similar levels of dephosphorylated FOXO were found for w¹¹¹⁸, K33 and hsNF1/+; K33, verified by Image J densitometry analysis in 3 experiments, one example shown. Panel B presents a bar graph showing total SOD and MnSOD activities of K33 and hsNF1/+; K33 flies, mean±SD of 5 experiments each involving 50 males and 50 females, total of 500 flies for each genotype. P=0.79 between their total SOD activities and P=0.15 between their MnSOD activities.

FIG. 15 schematically illustrates an exemplary behavior monitoring module.

Schematic figures are not necessarily to scale.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

A “trait” is a heritable characteristic. The term is used broadly herein to refer to any detectable phenotypic variation of a particular inherited character or attribute of an organism. Traits of particular interest in the invention include increased or decreased aging and longevity; additional traits of interest include stress resistance, up climbing/escape response, and mitochondrial respiration traits.

A “phenotype” is a trait or collection of traits that is/are observable in an individual or population. The trait can be quantitative (a quantitative trait, or QTL) or qualitative. For example, life span, stress resistance, up climbing/escape response, and mitochondrial respiration phenotypes can be monitored according to the methods and systems herein.

The term “adenylyl cyclase/cAMP/protein kinase A pathway” refers to the pathway in which adenylyl cyclase catalyzes the production of cAMP, which activates protein kinase A (PKA), which in turn acts on downstream components of the pathway, thereby increasing mitochondrial respiration, increasing ATP synthesis, and decreasing mitochondrial reactive oxygen species (ROS) production. A gene that regulates this pathway can increase or decrease expression, activity, or concentration of one or more components of the pathway or genes encoding components of the pathway (e.g., by affecting expression or activity of adenylyl cyclase or PKA, increasing or decreasing concentration of cAMP, or affecting expression or activity of a component downstream of PKA). Genes that regulate the pathway explicitly include genes whose polypeptide products (e.g., cAMP phosphodiesterase) increase or decrease expression, activity, or concentration of one or more components of the pathway or genes encoding components of the pathway. An adenylyl cyclase/cAMP/protein kinase A pathway can be modulated by increasing or decreasing the expression, activity, or concentration of one or more components of the pathway or of genes encoding one or more components of the pathway.

A “neurofibromatosis-1 gene” refers to a D. melanogaster neurofibromatosis-1 gene (e.g., as presented in Genbank accession no. L26501) or a homolog thereof, including an ortholog thereof from another species. Neurofibromatosis-1 genes also include genes whose protein products are homologous (e.g., orthologous) or substantially identical to the protein product of the D. melanogaster neurofibromatosis-1 gene (e.g., as presented in Genbank accession no. AAB58975). Other representative sequences include, but are not limited to, Genbank accession nos. AAB58976 and AAB58977.

An “adenylyl cyclase gene” refers to a D. melanogaster adenylyl cyclase gene (e.g., as presented in Genbank accession no. M81887) or a homolog thereof, including an ortholog thereof from another species. Adenylyl cyclase genes also include genes whose protein products are homologous (e.g., orthologous) or substantially identical to the protein product of the D. melanogaster adenylyl cyclase gene (e.g., as presented in Genbank accession no. AAA28844). Other representative sequences include, but are not limited to, Genbank accession no. P32870 (calcium/calmodulin sensitive).

A “cAMP phosphodiesterase gene” refers to a D. melanogaster cAMP phosphodiesterase gene (e.g., as presented in Genbank accession nos. X55167.1, X55168.1, X55169.1, X55170.1, X55171.1, X55172.1, X55173.1, X55174.1, and X55175.1) or a homolog thereof, including an ortholog thereof from another species. cAMP phosphodiesterase genes also include genes whose protein products are homologous (e.g., orthologous) or substantially identical to the protein product of the D. melanogaster cAMP phosphodiesterase gene (e.g., as presented in Genbank accession no. CAA38960). Examples of such orthologs include, but are not limited to, Genbank accession no. P14270.

A “protein kinase A gene” refers to a D. melanogaster protein kinase A gene (e.g., as presented in Genbank accession no. M18655.1, AAA28412.1, X16969.1, CAA34840.1, AE014134.5, AAF52797.1, AY069425.1, AAL39570.1, or C31751 (PKA catalytic subunit)) or a homolog thereof, including an ortholog thereof from another species. Protein kinase A genes also include genes whose protein products are homologous (e.g., orthologous) or substantially identical to the protein product of the D. melanogaster protein kinase A gene (e.g., as presented in Genbank accession no. P12370). Other representative sequences include, but are not limited to, Genbank accession nos. NP_{—}001014596, NP_{—}001014595, NP_{—}001014594, NP_{—}001014593, NP_{—}730573, NP_{—}730574, NP_{—}001014598, NP_{—}001014597, NP_{—}995672, NP_{—}730083, NP_{—}524189 NP_{—}788297, NP_{—}724860, NP_{—}733397, NP_{—}730576, NP_{—}524097, NP_{—}723479, NP_{—}524595, NP_{—}523671, and NP_{—}476977.

“Expression of a gene” or “expression of a nucleic acid” means transcription of DNA into RNA (optionally including modification of the RNA, e.g., splicing), translation of RNA into a polypeptide (possibly including subsequent modification of the polypeptide, e.g., posttranslational modification), or both transcription and translation, as indicated by the context.

As used herein, the term “gene” most generally refers to a combination of polynucleotide elements that, when operatively linked in either a native or recombinant manner, provide some product or function. Generally, the term gene is used broadly to refer to any nucleic acid associated with a biological function. The term gene is to be interpreted broadly herein, encompassing mRNA, cDNA, cRNA and genomic DNA forms of a gene. In some cases, a gene is heritable. In some aspects, genes comprise coding sequences (e.g., an “open reading frame” or “coding region”) necessary for the production of a polypeptide, while in other aspects, genes do not encode a polypeptide. Examples of genes that do not encode polypeptides include ribosomal RNA genes (rRNA) and transfer RNA (tRNA) genes.

The term gene can optionally encompass non-coding regulatory sequences that reside at a genetic locus. For example, in addition to a coding region of a nucleic acid, the term gene also encompasses the transcribed nucleotide sequences of the full-length mRNA adjacent to the 5′ and 3′ ends of the coding region. These noncoding regions are variable in size, and typically extend on both the 5′ and 3′ ends of the coding region. The sequences that are located 5′ and 3′ of the coding region and are contained on the mRNA are referred to as 5′ and 3′ untranslated sequences (5′ UT and 3′ UT). Both the 5′ and 3′ UT may serve regulatory roles, including translation initiation, post-transcriptional cleavage and polyadenylation.

In some aspects, the genomic form or genomic clone of a gene includes the sequences of the transcribed mRNA, as well as other non-transcribed sequences which lie outside of the transcript. The regulatory regions which lie outside the mRNA transcription unit are sometimes called 5′ or 3′ flanking sequences. A functional genomic form of a gene typically contains regulatory elements necessary for the regulation of transcription. For example, the term “promoter” is usually used to describe a DNA region, typically but not exclusively 5′ of the site of transcription initiation, sufficient to confer accurate transcription initiation. In some embodiments, a promoter is constitutively active, while in alternative embodiments, the promoter is conditionally active (e.g., where transcription is initiated only under certain physiological conditions). Exemplary non-constitutive promoters include tissue-specific, cell-type-specific, and inducible promoters. An “inducible” promoter is a promoter that is under environmental control and may be inducible or de-repressible; examples of environmental conditions that may effect transcription by inducible promoters include temperature, anaerobic conditions, or the presence of light or a specific compound. In some embodiments, the 3′ flanking region contains additional sequences which regulate transcription termination, sometimes caller terminator sequences. Generally, the term “regulatory element” refers to any genetic element that controls some aspect of the expression of nucleic acid sequences.

A “modulator” of a specified trait or phenotype is a compound that affects that trait or phenotype. For example, a modulator of aging or longevity can slow aging or increase longevity in an animal to which the modulator is administered. A modulator can, for example, partially or completely enhance or inhibit the activity of a protein (e.g., a catalytic activity of an enzyme, e.g., in an adenylyl cyclase/cAMP/PKA pathway), increase or decrease expression of a gene, increase or decrease concentration of a molecule of interest (e.g., cAMP), and/or the like. A modulator can be, e.g., a small molecule, a polypeptide, a nucleic acid, etc. Of particular interest are compounds that decrease physiological changes associated with aging and/or that increase longevity.

An “artificial mutation” is a mutation introduced by human intervention. Thus, an “artificially mutated” gene is a gene that has been mutated as a result of human intervention. For example, a gene can be artificially mutated using recombinant DNA techniques to alter, e.g., its coding region(s) and/or regulatory element(s), by insertion of a transposable element, or by exposing it to a chemical, ionizing radiation, or the like and then performing in vitro or in vivo selection for a desired mutated form of the gene. Artificial mutations include, but are not limited to, artificially introduced point mutations, insertions of one or more nucleotides (or amino acids, when referring to an encoded polypeptide), transposon insertions, and deletions of one or more nucleotides (or amino acids). Artificial mutation of a particular gene optionally also includes use of RNA interference or similar techniques (e.g., introduction or expression of a small interfering RNA, short hairpin RNA, etc.) to eliminate or reduce expression of the gene.

An “artificial disruption of expression of a gene” refers to reduction or elimination of expression of the gene, or alternatively to overexpression of the gene, achieved by human intervention. For example, expression can be artificially disrupted, without altering the endogenous gene's nucleic acid sequence, by introduction of antisense nucleic acid into the organism or cells thereof or by induction of RNA silencing in the organism or cells thereof. An artificial disruption can be produced, for example, by introducing an antisense nucleic acid, a short interfering RNA, a short hairpin RNA, a nucleic acid encoding an antisense nucleic acid, or a nucleic acid encoding a hairpin or other RNA that can be processed intracellularly to induce RNA silencing into the animal or cell(s) thereof. Preferably, the disruption is heritable.

As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In some aspects, the term “encode” describes the process of semi-conservative DNA replication, where one strand of a double-stranded DNA molecule is used as a template to encode a newly synthesized complementary sister strand by a DNA-dependent DNA polymerase.

In another aspect, the term “encode” refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

As used herein, the terms “heterologous” or “exogenous” as applied to polynucleotides or polypeptides refers to molecules that have been rearranged or artificially supplied to a biological system and are not in a native configuration (e.g., with respect to sequence, genomic position or arrangement of parts) or are not native to that particular biological system. The terms indicate that the relevant material originated from a source other than the naturally occurring source, or refers to molecules having a non-natural configuration, genetic location or arrangement of parts. The terms “exogenous” and “heterologous” are sometimes used interchangeably with “recombinant.”

The term “recombinant” in reference to a nucleic acid or polypeptide indicates that the material (e.g., a recombinant nucleic acid, gene, polynucleotide, polypeptide, etc.) has been altered by human intervention. Generally, the arrangement of parts of a recombinant molecule is not a native configuration, or the primary sequence of the recombinant polynucleotide or polypeptide has in some way been manipulated. The alteration to yield the recombinant material can be performed on the material within or removed from its natural environment or state. For example, a naturally occurring nucleic acid becomes a recombinant nucleic acid if it is altered, or if it is transcribed from DNA which has been altered, by means of human intervention performed within the cell from which it originates; a gene sequence open reading frame is recombinant if that nucleotide sequence has been removed from its natural context and cloned into any type of artificial nucleic acid vector; and a polypeptide or protein is recombinant when it is produced by expression of a recombinant nucleic acid. The term recombinant (or “transgenic”) can also refer to an organism that harbors recombinant material. Protocols and reagents to produce recombinant molecules, especially recombinant nucleic acids, are common and routine in the art (see, e.g., Maniatis et al. (eds.), Molecular Cloning: A Laboratory Manual, Cold Spring Harbor Laboratory Press, NY, [1982]; Sambrook et al. (eds.), Molecular Cloning: A Laboratory Manual, Second Edition, Volumes 1-3, Cold Spring Harbor Laboratory Press, NY, [1989]; and Ausubel et al. (eds.), Current Protocols in Molecular Biology, a joint venture between Greene Publishing Associates, Inc. and John Wiley & Sons, Inc., supplemented through 2007).

The term “animal” refers to an invertebrate or vertebrate animal. In some aspects, animals include humans, while other aspects relate only to non-human animals. Exemplary non-human animals include, but are not limited to, insects (e.g., Drosophila, including Drosophila melanogaster), nematodes (e.g., Caenorhabditis elegans), mammals, non-human primates, rodents (e.g., mice, rats, and hamsters), stock and domesticated animals (e.g., pigs, cows, sheep, horses, cats, and dogs), and birds.

The term “insect” refers to organisms belonging to the phylogenetic class Insecta.

A variety of additional terms are defined or otherwise characterized herein.

### DETAILED DESCRIPTION

Neurofibromin, the protein product of the NF1 gene, is a tumor suppressor, and its GTPase-activating protein (GAP) domain negatively regulates Ras activity; when NF1 function is compromised, Ras becomes hyperactive. Neurofibromin also positively regulates the G protein-stimulated and Ca²⁺/calmodulin-sensitive adenylyl cyclase in both D. melanogaster and mice². Consequently, inactivation of NF1 results in upregulation of the Ras/Raf pathway⁹ and downregulation of adenylyl cyclase/cAMP/protein kinase A (PKA) signaling⁶⁻⁸.

Most studies on the pathophysiology NF1 have focused on the Ras/Raf pathway^{2,4,5} because of its well-established role in tumorigenesis⁹. However, recent research has demonstrated the importance of neurofibromin regulation of adenylyl cyclase, cAMP and PKA activity in the etiology of impaired learning and related electrophysiological abnormalities in D. melanogaster NF1 mutants^{6-8,10}.

Extensive studies are reported herein on both the inactivation and the overexpression of the NF1 gene in D. melanogaster. The examples hereinbelow demonstrate that neurofibromin deficiency shortens life span and increases sensitivity to oxidative stress, whereas increased neurofibromin extends life span and improves resistance to oxidative and thermal stress. These phenotypes are shown to be mediated by modulation of the adenylyl cyclase/cAMP/PKA pathway and associated with changes in mitochondrial NADH-linked respiration and reactive oxygen species (ROS) production. Finally, a causal role for cAMP and mitochondrial ROS production in determining life span is established, cAMP analogs are demonstrated to extend life spans of wild-type flies, and catalytic antioxidant drugs are shown to ameliorate the life reduction of the NF1 mutants.

Accordingly, one aspect of the present invention provides methods for treating NF1 disorders by administration of catalytic antioxidant to patients with such disorders. Methods and systems for identifying appropriate modulators are also described.

The links identified between NF1, the adenylyl cyclase/cAMP/protein kinase A pathway, mitochondrial function, and life span provide additional aspects of the present invention, including, without limitation, methods of and systems for screening for modulators of aging (also referred to as senescence), longevity, or life span and methods for regulating longevity. Related transgenic animals are also described.

### Methods of Screening for Modulators of Aging or Longevity

One general class of embodiments provides methods of screening for a modulator of aging or longevity. In the methods, a non-human animal with an artificial mutation in, or an artificial disruption of expression of, a gene that encodes a component of or that regulates an adenylyl cyclase/cyclic AMP/protein kinase A pathway in the animal, wherein the mutation or disruption is correlated with an aging or longevity trait for the non-human animal, is provided. The modulator is administered to the non-human animal. An effect of the modulator on a phenotype of the non-human animal, wherein the phenotype is correlated to said mutation or disruption, is monitored.

Optionally, the mutation (or disruption of expression) is in a gene that encodes a component of the adenylyl cyclase/cyclic AMP/protein kinase A pathway, for example, in an adenylyl cyclase gene or a protein kinase A gene. As other examples, the mutation (or disruption of expression) can be in a gene that regulates the pathway, for example, in a neurofibromatosis-1 gene (as noted above, NF1 positively regulates the pathway) or a cAMP phosphodiesterase gene (cAMP phosphodiesterase degrades cAMP). In one class of embodiments, the mutation results in inactivation of the gene, substantially reducing or entirely eliminating its expression or activity of its protein product (e.g., reducing expression or activity by at least 50%, at least 75%, or at least 90%, or rendering it undetectable). In other embodiments, the mutation results in overexpression of the gene. Such overexpression can result, for example, in the mRNA or protein product of the gene being present in an animal (or a tissue or cell or extract thereof) at an amount that is at least 2×, at least 5×, at least 10×, at least 50×, or even at least 100× normal for that animal, tissue, or cell type (including expression in an animal, tissue, or cell not normally expressing the gene). The animal is optionally homozygous for the mutation, or it can bear one allele with the mutation and another allele with a different mutation in the gene, or it can be heterozygous for the mutant allele and a wild-type allele.

Suitable non-human animals include, but are not limited to, nematodes (e.g., Caenorhabditis elegans), mammals, non-human primates, rodents (e.g., mice, rats, and hamsters), stock and domesticated animals (e.g., pigs, cows, sheep, horses, cats, and dogs), and birds. In one class of embodiments, the non-human animal is an insect, for example, a Drosophila such as a Drosophila melanogaster.

The modulator can be administered by feeding it to the non-human animal, or it can be administered through another route such as injection, topical application, transdermal application, or the like. The modulator is optionally administered in a single dose or in multiple doses, over hours, days, weeks, months, or years, depending, e.g., on the normal life span of the type of animal.

Aging and longevity traits of interest include, but are not limited to, aging, longevity (life span), stress resistance, up climbing/escape response, and mitochondrial respiration traits. The aging or longevity trait correlated with the mutation or disruption can be the same as, included in, or different from the phenotype or trait to be monitored for effectiveness of the modulator.

Any of a variety of different assays can be employed, depending on the phenotype of interest. For example, in one class of embodiments, the phenotype is a life span phenotype, and monitoring the effect of the modulator comprises performing a longevity assay that measures the life span of the animal in presence of the modulator. In another exemplary class of embodiments, the phenotype is a stress resistance phenotype, and monitoring the effect of the modulator involves performing a stress resistance assay that measures stress resistance of the animal in presence of the modulator. In this class of embodiments, the stress resistance phenotype optionally comprises reduced resistance to heat or oxidative stress as compared to an isogenic or near isogenic animal that lacks the mutation or disruption, and monitoring the effect of the modulator comprises detecting increased resistance to heat or oxidative stress caused by the modulator.

In another exemplary class of embodiments, the phenotype comprises a physical activity or locomotion phenotype, and monitoring the effect of the modulator comprises performing a physical activity assay that measures physical activity of the animal in presence of the modulator. For example, the animal can be an insect, and the physical activity assay can include measuring up climbing/escape response activity of the insect.

In yet another exemplary class of embodiments, the phenotype comprises an alteration in mitochondrial respiration, and monitoring the effect of the modulator comprises performing a mitochondrial respiration activity assay that measures mitochondrial respiration in cells or tissues of the animal, or in an extract thereof, in presence of the modulator. In a related class of embodiments, the phenotype comprises a mitochondrial respiration trait and monitoring the effect of the modulator comprises performing a mitochondrial respiration activity assay that measures mitochondrial respiration in the animal, in cells or tissues of the animal, or in an extract thereof, after administration of the modulator.

Other exemplary phenotypes that can be monitored, e.g., as described herein, include (a.) cAMP concentration in the animal, in cells or tissues of the animal, or in an extract thereof, (b.) complex I activity in the animal, in cells or tissues of the animal, or in an extract thereof, (c.) citrate synthase activity in the animal, in cells or tissues of the animal, or in an extract thereof, (d.) mitochondrial ROS production in the animal, in cells or tissues of the animal, or in an extract thereof, (e.) mitochondrial respiratory control ratio (state III O₂ consumption rate/state IV O₂ consumption rate) in the animal, in cells or tissues of the animal, or in an extract thereof, (f.) ATP production rate when metabolizing NADH-linked substrates in the animal, in cells or tissues of the animal, or in an extract thereof, (g.) aconitase activity in the animal, in cells or tissues of the animal, or in an extract thereof, (h.) superoxide dismutase or catalase activity in the animal, in cells or tissues of the animal, or in an extract thereof, and (i.) reproductive capacity of the animal.

Optionally, in any of the above embodiments, the phenotype of the animal in the presence of the modulator is compared to that of an isogenic or nearly isogenic animal in the absence of the modulator.

Exemplary modulators include, but are not limited to, a cAMP analog, an antioxidant, a catalytic antioxidant (an antioxidant that can, e.g., destroy multiple ROS without itself becoming inactivated), or a metalloporphyrin catalytic antioxidant. Examples of such modulators are described herein, and additional suitable potential modulators are known in the art or can readily be produced using techniques (e.g., chemical synthesis techniques) well known in the art. See, for example, the catalytic antioxidants, macrocyclics, salens, and porphyrins (e.g., Mn metalloporphyrins) described in Day (2004) “Catalytic antioxidants: a radical approach to new therapeutics” Drug Discovery Today 9:557-566, Melov et al. (2001) “Lifespan extension and rescue of spongiform encephalopathy in superoxide dismutase 2 nullizygous mice treated with superoxide dismutase-catalase mimetics” J Neurosci 21:8348-8353, Milano and Day (2000) “A catalytic antioxidant metalloporphyrin blocks hydrogen peroxide-induced mitochondrial DNA damage” Nucl Acids Res 28:968-973, Melov et al. (2000) “Extension of life-span with superoxide dismutase/catalase mimetics” Science 289:1567-1569, and U.S. Pat. Nos. 6,479,477, 6,544,975, and 6,916,799. A modulator can be, e.g., a small molecule (e.g., a compound with a molecular weight less than 1000 daltons), a polypeptide, a nucleic acid (e.g., an siRNA), etc.

Potential modulator libraries to be screened are available. These libraries are optionally random or targeted. Targeted libraries include those designed using any form of a rational design technique that selects scaffolds or building blocks to generate combinatorial libraries. These techniques include a number of methods for the design and combinatorial synthesis of target-focused libraries, including morphing with bioisosteric transformations, analysis of target-specific privileged structures, and the like. In general, where information regarding structure of adenylyl cyclase/cAMP/protein kinase A pathway genes or gene products is available, likely binding partners can be designed, e.g., using flexible docking approaches, or the like. Similarly, random libraries exist for a variety of basic chemical scaffolds. In either case, many thousands of scaffolds and building blocks for chemical libraries are available, including those with polypeptide, nucleic acid, carbohydrate, and other backbones. Commercially available libraries and library design services include those offered by Chemical Diversity (San Diego, Calif.), Affymetrix (Santa Clara, Calif.), Sigma (St. Louis Mo.), ChemBridge Research Laboratories (San Diego, Calif.), TimTec (Newark, Del.), Nuevolution A/S (Copenhagen, Denmark) and many others.

Before testing in a whole animal screen, potential modulators are optionally pre-screened in a cell-free assay (e.g., for binding to NF1 or an adenylyl cyclase/cAMP/protein kinase A pathway component, for modulating activity of NF1 or a pathway component, or for effect on mitochondrial respiration) and/or a cell-based assay (e.g., by contacting a cell, optionally one with an artificial mutation in or an artificial disruption of expression of a gene that encodes a component of or that regulates an adenylyl cyclase/cyclic AMP/protein kinase A pathway, with the modulator and then measuring mitochondrial respiration, gene expression, protein activity, or another relevant phenotype as described above in the cell).

In a screen for modulators, optionally a panel of different modulators or potential modulators (i.e., two or more) are administered to different animals, the affect of each modulator on the phenotype is monitored, and one or more modulators having the desired effect are identified, for example, by identifying modulators that increase life span, stress resistance, physical activity, mitochondrial respiration, etc.

Another general class of embodiments provides methods of screening for a modulator that increases life span. The methods include the steps of administering a putative modulator to a non-human animal, and testing for increased neurofibromin expression or activity in the animal following administration of the modulator, wherein increased neurofibromin expression or activity in the animal correlates with increased life span. Neurofibromin expression can be detected using techniques described hereinbelow or similar techniques well known in the art.

Essentially all of the features noted above apply to this class of embodiments as well, as relevant; for example, with respect to type of non-human animal, exemplary modulators, and/or the like. It is worth noting that the non-human animal optionally has a mutation (naturally occurring or artificial) in a gene, or an artificial disruption of expression of a gene, that encodes a component of or that regulates an adenylyl cyclase/cyclic AMP/protein kinase A pathway in the animal.

Yet another general class of embodiments provides methods of screening for a modulator that increases life span. In this class of embodiments, the methods include administering the modulator to a non-human animal and testing for changes in adenylyl cyclase/cyclic AMP/protein kinase A pathway component expression, activity, or concentration. For example, changes in adenylyl cyclase or protein kinase A activity or expression can be monitored following administration of the modulator, or cAMP concentration can be determined and compared before and after administration of the modulator.

Essentially all of the features noted above apply to this class of embodiments as well, as relevant; for example, with respect to type of non-human animal, exemplary modulators, presence of natural or artificial mutations or disruptions, and/or the like.

### Systems for Screening for Modulators of Aging or Longevity

Systems for screening for modulators of aging or longevity are also a feature of the invention. For example, one class of embodiments provides a system for screening for a modulator compound that modulates an aging related behavioral phenotype that comprises an array of non-human animals in containers (e.g., vials, tubes, boxes, cages, etc. as appropriate for the type of animal), a behavior monitoring module that monitors the behavioral phenotype of the animals in the containers in the presence of the modulator, and a correlation module that correlates behavior of the animal to aging or life span.

Arrays of the invention can be standard gridded arrays that have a logical spatial relationship among members of the array, e.g., vials of Drosophila in a rack. The array can also be a “logical array” in which the members of the array are linked by a look-up table that tracks array members, such as individual vials. In the later series of embodiments, standard tracking software can be used to track vial positions, and different logical arrays, e.g., sets of vials, can be treated with one or more different modulators. In the case where the arrays are arranged in a standard spatial arrangement, the entire array, or selected members, can be treated with one or more modulators and the effects observed.

In one aspect, the behavior monitoring module monitors physical activity of the animals, or arrays thereof, e.g., climbing/escape response behavior. Essentially all of the features noted above apply to this class of embodiments as well, as relevant; for example, with respect to type of non-human animal, exemplary modulators, presence of natural or artificial mutations or disruptions in the animal, and/or the like.

A related class of embodiments provides a system for screening for modulator compounds that modulate an aging related behavioral trait in which the system includes an array of insects in containers and a behavior monitoring module that monitors physical activity of the insects in the array following administration of the modulator compounds. In one embodiment, the system comprises an automated shaker or tapper that consistently shakes or taps the containers of the array. Again, essentially all of the features noted above apply to this class of embodiments as well, as relevant; for example, with respect to type of non-human animal, exemplary modulators, presence of natural or artificial mutations or disruptions in the animal, and/or the like.

Optionally, the system will include (e.g., in a correlation module) system instructions that correlate behavior of the animals with a predicted aging or life span phenotype, e.g., instructions that correlate increased physical activity, increased (or decreased) escape response, or decreased (or increased) recovery time after heat stress with increased (or decreased) life span or decreased (or increased) aging. The system instructions can compare detected information as to behavior (e.g., physical activity level) with a database that includes correlations between behaviors and the relevant phenotypes. This database can be multidimensional, thereby including higher-order relationships between combinations of behaviors or other information (e.g., cAMP concentration, mitochondrial respiration, etc.) and the relevant phenotypes. These relationships can be stored in any number of look-up tables, e.g., taking the form of spreadsheets (e.g., Excel™ spreadsheets) or databases such as an Access™, SQL™, Oracle™, Paradox™, or similar database. The system can include provisions for inputting animal-specific information regarding behavior information, e.g., through an automated or user interface, and for comparing that information to the look up tables.

The correlation module can include software that tracks and analyzes data relationships. For example, Partek Incorporated (St. Peters, Mo.; www (dot) partek (dot) corn) provides software for pattern recognition (e.g., which provide Partek Pro 2000 Pattern Recognition Software) which can be applied to, e.g., principle component analysis, genetic algorithms for multivariate data analysis, interactive visualization, variable selection, and neural & statistical modeling. Relationships can be analyzed, e.g., by Principal Components Analysis (PCA) mapped scatterplots and biplots, Multi-Dimensional Scaling (MDS) mapped scatterplots, Star plots, etc. The software of the system can be heuristic in nature, e.g., by including neural networks or statistical methods to detect and analyze data relationships. For example, neural net approaches can be coupled to genetic algorithm-type programming for heuristic development of a modulator-trait data space model. For example, NNUGA (Neural Network Using Genetic Algorithms) is an available program (e.g., on the world wide web at cs (dot) bgu (dot) ac (dot) il/˜omri/NNUGA which couples neural networks and genetic algorithms. An introduction to neural networks can be found, e.g., in Kevin Gurney (1999) An Introduction to Neural Networks, UCL Press, 1 Gunpowder Square, London EC4A 3DE, UK. and on the world wide web at shef (dot) ac (dot) uk/psychology/gurney/notes/index (dot) html. Additional useful neural network references include, e.g., Christopher M. Bishop (1995) Neural Networks for Pattern Recognition Oxford Univ Press; ISBN: 0198538642; Brian D. Ripley, N. L. Hjort (Contributor) (1995) Pattern Recognition and Neural Networks Cambridge University Press (Short); ISBN: 0521460867. The correlation module can include any available statistical tool for detecting, correlating, predicting or analyzing modulator data, including multidimensional data as noted above.

For example, the system instructions can include software that accepts information associated with any detected behavior information, e.g., an indication that a subject with the relevant behavior has a particular phenotype. This software can be heuristic in nature, using such inputted associations to detect correlations, or to improve the accuracy of the look up tables and/or interpretation of the look up tables by the system. A variety of such approaches, including principle component analysis, neural networks, genetic algorithms, Markov modeling, and other statistical analysis are known in the art and can be incorporated into the system software.

The invention includes behavior monitoring modules for monitoring one or more behavioral phenotypes of the animals (e.g., physical activity). For example, the behavior monitoring module can include one or more marked tubes, vials, or boxes for quantitating locomotion or up climbing/escape response behavior. The module can include components for eliciting the behavior, e.g., an automated shaker or tapper, a heat source for inducing stress response, and/or the like. Behavior can be monitored manually, or monitoring can be automated, for example, by automated counting of animals passing through an infrared beam as they demonstrate up climbing/escape response behavior. Such automated behavior detection apparatus can be included in the system.

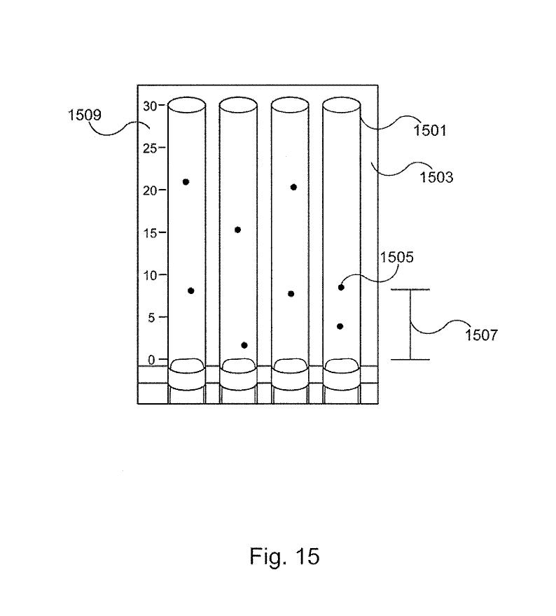

An exemplary behavior monitoring module for monitoring physical activity (specifically, up climbing behavior) and/or for monitoring recovery after heat stress is schematically illustrated in FIG. 15. In this example, an array of Drosophila melanogaster 1505 in vertical tubes 1501 is provided. While two flies per tube are illustrated for simplicity, it will be evident that essentially any convenient number can be included (e.g., ten). Similarly, four tubes are illustrated, but other numbers can be employed. Tubes 0.5 cm in diameter and 30 cm in height have proved to be convenient, although it will be evident that other dimensions are readily employed. Tubes 1501 are attached to backboard 1503, which facilitates simultaneous tapping of the tubes. Reference marks 1509 are optionally included on backboard 1503 to facilitate quantitation of up climbing behavior. Heat stress can be achieved by placing the device in a temperature controlled chamber. Behavior of the flies in the module is monitored. For example, locomotive index can be determined as described in the Examples section hereinbelow. As another example, climbing velocity can be calculated by tapping the tubes until the flies are located at the bottom, allowing the flies to climb up the tubes, and measuring the height to which each travels (i.e., distance 1507) in a given time (e.g., after 30 and 60 seconds). Climbing velocity is then calculated as velocity (cm/s)=distance (cm)/time (s). The average climbing velocity of modulator-treated flies in one tube is optionally compared to that of untreated flies in another tube.

The animals whose behavior is to be analyzed are optionally part of the system, or they can be considered separate from it. In one aspect, the animals are insects, e.g., Drosophila melanogaster. Other useful model organisms include nematodes (e.g., C. elegans), invertebrates, and rodents.

Optionally, system components for interfacing with a user are provided. For example, the systems can include a user viewable display for viewing an output of computer-implemented system instructions, user input devices (e.g., keyboards or pointing devices such as a mouse) for inputting user commands and activating the system, etc. Typically, the system of interest includes a computer, wherein the various computer-implemented system instructions are embodied in computer software, e.g., stored on computer readable media.

In addition to statistical software, standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Sequel™, Oracle™, Paradox™) can be adapted to the present invention by inputting a character string corresponding to a modulator, a behavior or other trait herein, or to an association between a modulator or behavior or other trait and a phenotype. Suitable software can also easily be constructed by one of skill using a standard programming language such as Visualbasic, Fortran, Basic, Java, or the like.

As noted, systems can include a computer with an appropriate database and a behavior or correlation of the invention. Data sets entered into the software system comprising any of the behaviors or behavior-phenotype correlations herein can be a feature of the invention. The computer can be, e.g., a PC (Intel x86 or Pentium chip-compatible DOS™, OS2™ WINDOWS™ WINDOWS NT™, WINDOWS95™, WINDOWS98™, WINDOWS2000, WINDOWSME, WINDOWS VISTA, or LINUX based machine, a MACINTOSH™, Power PC, or a UNIX based (e.g., SUN™ work station or LINUX based machine) or other commercially common computer which is known to one of skill.

### Methods of Treating NF1 Disorders and Regulating Longevity

In one aspect, the invention includes methods of treating an NF1 disorder. In the methods, a catalytic antioxidant is administered to a patient (typically, a human patient) suffering from the disorder. The NF1 disorder, e.g., neurofibromatosis-1, can be diagnosed as established in the art. NF1 is associated with a spectrum of tissue-specific and systemic manifestations including café-au-lait spots, benign as well as metastatic tumors particularly neurofibromas, developmental abnormalities, and learning disabilities. Optionally, the catalytic antioxidant to be administered is a metalloporphyrin.

Another aspect of the invention provides methods for regulating (e.g., increasing) longevity of an animal. In these embodiments, an adenylyl cyclase/cyclic AMP/protein kinase A pathway in the animal is modulated. A related aspect provides methods of regulating (e.g., increasing) longevity of an animal; in this aspect, neurofibromin expression or activity in the animal is modulated. Such modulation can be effected, for example, by administration of a modulator, overexpression or inhibition of expression of NF1, etc. In either aspect, the animal can be a human, or it can be a non-human animal (e.g., an insect). In some embodiments, the animal comprises a mutation in, or an artificial disruption of expression of, one or more of a neurofibromatosis-1 gene, an adenylyl cyclase gene, a cAMP phosphodiesterase gene, and a protein kinase A gene (e.g., a natural or artificial mutation). The methods optionally include increasing neurofibromin expression or activity in the animal. In one class of embodiments, the methods include administering a longevity modulator to the animal over an extended period of time, e.g., over hours, days, weeks, months, or years, depending, e.g., on the normal life span of the type of animal.

The methods described herein, as well as modulators identified using the methods and screening systems herein, are relevant not only to life span and longevity but to all age-related metabolic and degenerative diseases, activity and muscle strength, cancer, and other indicators of wellbeing. As just one example, since NF1 is a putative tumor suppressor gene in mammals, screening NF1-deficient Drosophila for drugs that increase longevity, stress resistance and/or physical capacity can identify cancer therapeutics useful in mammals, including humans. Any modulator identified in the methods herein can be screened for antitumor or anticancer activity in any available cancer model, including cell-based and model-animal models of cancer. The observation that catalytic antioxidants restored the life span of the NF1 mutant flies to normal identifies mitochondrially-targeted antioxidants as a potent new strategy for treating tumor cell proliferation.

### Therapeutic Administration of Modulators

Various aspects of the invention involve administration of a modulator to a human patient or non-human animal. In embodiments in which a modulator (including a catalytic antioxidant) is administered, particularly to a human, for example, to treat an NF1 disorder or an age-related disease or symptom or to increase longevity, compositions for administration typically comprise a therapeutically effective amount of the modulator (i.e., an amount that is effective for preventing, ameliorating, or treating a disease or disorder, preventing or ameliorating physiological effects of aging, extending life span, or the like) and a pharmaceutically acceptable carrier or excipient. Such a carrier or excipient includes, but is not limited to, saline, buffered saline, dextrose, water, glycerol, ethanol, and/or combinations thereof. Pharmaceutically acceptable carriers are determined in part by the particular composition being administered, as well as by the particular method used to administer the composition. Accordingly, there is a wide variety of suitable formulations of pharmaceutical compositions of the present invention.

Therapeutic compositions comprising one or more modulators of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

Compositions can be administered by a number of routes including, but not limited to: oral, intravenous, intraperitoneal, intramuscular, transdermal, subcutaneous, topical, sublingual, or rectal administration. Such administration routes and appropriate formulations are generally known to those of skill in the art.

The compositions, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials.

The dose administered to a patient, in the context of the present invention, is sufficient to effect a beneficial, e.g., prophylactic and/or therapeutic, response in the patient over time. The dose is determined, e.g., by the efficacy of the particular compound or other formulation and the condition of the patient, as well as the body weight or surface area of the patient to be treated. The size of the dose is also determined by the existence, nature, and extent of any adverse side-effects that accompany the administration of a particular formulation in a particular patient. In determining the effective amount of the modulator or formulation to be administered in the treatment of disease or extension of life span, the physician evaluates such factors as circulating plasma levels of the modulator, formulation toxicities, and progression of the relevant disease.

For administration, formulations of the present invention are administered at a rate determined by the LD-50 of the relevant formulation, and/or observation of any side-effects of the modulators of the invention at various concentrations, e.g., as applied to the mass or topical delivery area and overall health of the patient. Administration can be accomplished via single or divided doses, and can be continued for an extended period.

If a patient undergoing treatment develops fevers, chills, or muscle aches, he/she receives the appropriate dose of aspirin, ibuprofen, acetaminophen or other pain/fever controlling drug. Patients who experience reactions to the compositions, such as fever, muscle aches, and chills are premedicated 30 minutes prior to the future infusions with either aspirin, acetaminophen, or, e.g., diphenhydramine. Meperidine is used for more severe chills and muscle aches that do not quickly respond to antipyretics and antihistamines. Treatment is slowed or discontinued depending upon the severity of the reaction.

### Transgenic Animals

Transgenic animals related to or of use in the methods and systems of the invention are also featured. Accordingly, one general class of embodiments provides a transgenic non-human animal comprising a knock out or knock down mutation in, or an artificial and preferably heritable disruption of expression of, one or more copies of an NF1, an adenylyl cyclase, a cAMP phosphodiesterase, or a PKA gene in the genome of the animal, wherein the animal further comprises a recombinant NF1, adenylyl cyclase, cAMP phosphodiesterase, or PKA gene. The mutation or disruption typically substantially reduces or entirely eliminates expression of the gene or activity of its protein product (e.g., reducing expression or activity by at least 50%, at least 75%, or at least 90%, or rendering it undetectable). The recombinant gene is optionally under control of an endogenous promoter, a heterologous promoter, and/or an inducible promoter (e.g., a heat shock promoter), and is optionally from the same and/or a different species. Exemplary non-human animals have been described above.

As a few specific examples, in one class of embodiments, the animal is an insect, the gene is an NF1 gene, and the recombinant NF1 gene is under the control of a heterologous inducible promoter. Similarly, in one class of embodiments, the animal is an insect, the gene is an adenylyl cyclase gene, and the recombinant adenylyl cyclase gene is under the control of a heterologous inducible promoter. In one class of embodiments, the animal is an insect, the gene is a PKA gene, and the recombinant PKA gene is under the control of a heterologous inducible promoter.

Methods of making transgenic animals that have knock out or knock down mutations and/or that express heterologous genes are well known in the art. In general, such a transgenic animal is typically one that has had appropriate genes (or partial or recombinant genes, e.g., comprising coding sequences coupled to a promoter) introduced into one or more of its cells artificially. For example, a DNA can be integrated randomly, e.g., by injecting it into the pronucleus of a fertilized ovum such that the DNA can integrate anywhere in the genome without need for homology between the injected DNA and the host genome. P-element mediated transduction in Drosophila provides one such classical and well understood system. As another example, targeted insertion can be accomplished, e.g., by introducing the heterologous DNA, e.g., into embryonic stem (ES) cells, and selecting for cells in which the heterologous DNA has undergone homologous recombination with homologous sequences of the cellular genome. This is common particularly in non-human mammalian transgenic systems, e.g., in making transgenic rodents such as transgenic mice.

As noted, one common use of targeted insertion of DNA is to make knock-out mice. These are useful in the present invention in a variety of contexts, e.g., as targets for modulator studies. Similarly, transgenic animals that comprise deletions of natural NF-1 or other adenylyl cyclase/cAMP/protein kinase A pathway genes can also include targeted insertion of corresponding human genes. This provides an improved model system that is more highly correlated with the human pathway.

In these approaches, typically, homologous recombination is used to insert a selectable gene (e.g., an antibiotic resistance gene or another positive selectable marker) driven by a constitutive promoter into an essential exon of the gene that one wishes to disrupt (e.g., the first coding exon). To accomplish this, the selectable marker is flanked by large stretches of DNA that match the genomic sequences surrounding the desired insertion point (typically, there are several kilobases of homology between the heterologous and genomic DNA). Once this construct is electroporated into ES cells, the cells' own machinery performs the homologous recombination. To make it possible to select against ES cells that incorporate DNA by non-homologous recombination (e.g., random insertion), it is common for targeting constructs to include a negatively selectable gene outside the region intended to undergo recombination (typically the gene is cloned adjacent to the shorter of the two regions of genomic homology). Because DNA lying outside the regions of genomic homology is lost during homologous recombination, cells undergoing homologous recombination cannot be selected against, whereas cells undergoing random integration of DNA often can. A commonly used gene for negative selection is the herpes virus thymidine kinase gene, which confers sensitivity to the drug gancyclovir.

Following positive selection and negative selection if desired, ES cell clones are screened for incorporation of the construct into the correct genomic locus. Typically, one designs a targeting construct so that a band normally seen on a Southern blot or following PCR amplification becomes replaced by a band of a predicted size when homologous recombination occurs. Since ES cells are diploid, only one allele is usually altered by the recombination event so, when appropriate targeting has occurred, one usually sees bands representing both wild type and targeted alleles.

The embryonic stem (ES) cells that are used for targeted insertion are derived from the inner cell masses of blastocysts (early mouse embryos). These cells are pluripotent, meaning they can develop into any type of tissue.

Once positive ES clones have been grown up and frozen, the production of transgenic animals can begin. Donor females are mated, blastocysts are harvested, and several ES cells are injected into each blastocyst. Blastocysts are then implanted into a uterine horn of each recipient. By choosing an appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring. It will be evident that analogous techniques can be used to introduce essentially any heterologous gene of interest, instead of or in addition to knocking out an endogenous gene in the mouse.

Methods for making transgenic insects, particularly Drosophila melanogaster, have also been described. For example, use of P elements to make transgenic flies is well known in the art. P elements can be used, e.g., to knock out or knock down expression of an endogenous gene and/or to introduce a heterologous gene. Typically, the gene of interest is placed between P element ends, usually within a plasmid, and injected into pre-blastoderm embryos in the presence of transposase. The P element then transposes from the plasmid to a random chromosomal site, carrying the gene with it. The P element typically also carries a second gene for convenient identification of transformants. A visible marker such as an eye color gene is generally preferred, although other markers can be employed, e.g., a selectable marker such as neomycin resistance. The transposase can be provided, for example, by binding a purified transposase protein to the element prior to injection, by coinjecting a transposase-encoding helper plasmid, or most typically by injecting directly into embryos that have an endogenous transposase. The transposase-bearing chromosome can be marked with a dominant mutation, such that stable transformants lacking the transposase gene can be selected among the progeny.

A variety of P element vectors are available in the art, including vectors to facilitate expression of the gene of interest in particular tissues, at particular times in development, or upon induction by elevated temperature, for example. Additional vectors can readily be constructed or modified as needed. Available vectors include those encoding the FLP site-specific recombinase and bearing its target site FRT, which can be used to generate somatic mosaics by site-specific recombination. P element mediated transformation can also be employed to achieve gene replacement by making use of P-induced double strand breaks. In addition, a P element can be mobilized such that insertion occurs at a large number of random sites. Progeny bearing such insertions are then screened to identify lines in which the element is inserted within a desired gene, e.g., to reduce or eliminate expression of the gene.

Similar techniques enable construction of transgenic animals of other species. Additional details are available in the art. See, e.g., Ashburner et al. (2004) Drosophila: A Laboratory Handbook, 2^{nd} edition Cold Spring Harbor Laboratory Press, Greenspan (2004) Fly Pushing: The Theory and Practice of Drosophila Genetics, 2^{nd} edition Cold Spring Harbor Laboratory Press, Sullivan et al. (eds) (2000) Drosophila Protocols Cold Spring Harbor Laboratory Press, Roberts (ed) (1998) Drosophila: A Practical Approach Oxford University Press, USA, Schepers (2005) RNA Interference in Practice: Principles, Basics, and Methods for Gene Silencing in C. elegans, Drosophila, and Mammals Wiley-VCH, Nagy et al. (eds) (2002) Manipulating the Mouse Embryo: A Laboratory Manual, 3rd edition Cold Spring Harbor Laboratory Press, Tymms and Kola (eds) (2001) Gene Knockout Protocols (Methods in Molecular Biology) Humana Press, Hofker and van Deursen (eds) (2002) Transgenic Mouse Methods and Protocols (Methods in Molecular Biology) Humana Press, Hope (ed) (2002) C. elegans: A Practical Approach (Practical Approach Series) Oxford University Press, USA, and Strange (ed) (2006) C. elegans: Methods and Applications (Methods in Molecular Biology) Humana Press.

As with the murine system described above, human genes in the relevant pathway, e.g., NF-1 or a gene in the adenylyl cyclase/cAMP/protein kinase A pathway, can be introduced into Drosophila (or any other model organism) to more accurately screen for modulators of the human genes or proteins, and to study human gene function.

Transgenic animals are a useful tool for studying gene function and testing modulators or potential modulators. For example, a variety of transgenic animals useful in the screening systems and methods of the present invention have been described in detail above. As an additional example, human (or other selected) homolog genes can be introduced in place of the endogenous NF1, adenylyl cyclase/cAMP/protein kinase A pathway, and/or other related genes of a laboratory animal, making it possible to study function of the human (or other) polypeptide or complex in the easily manipulated and studied laboratory animal. It will be appreciated that there is not always precise correspondence between protein structure or function of different animals, making the ability to study the human or other gene of interest particularly useful when developing clinical candidate modulators. Although similar genetic manipulations can be performed in tissue culture, the interaction of NF1, adenylyl cyclase, protein kinase A, and other components of the pathway in the context of an intact organism can provide a more complete and physiologically relevant picture of function than could be achieved in non-cell based assays or simple cell-based screening assays. Accordingly, transgenic animals are particularly useful when analyzing modulators identified in high throughput in vitro (e.g., cell-free and/or cell-based) systems. As another advantage, in higher organisms with at least two homolog genes, compounds that selectively induce or inhibit the activity or expression of one homolog protein and not another may be identified in assays using pairs or groups of such transgenic animals (or, similarly, pairs of transgenic cell lines in cell-based assays) each only expressing one homolog gene and comparing the effect of the compound on each organism (or cell line).

Other methods for reducing or eliminating expression or activity, e.g., by inducing artificial mutations (e.g., point, deletion, or insertion mutations) in a gene and screening for individuals with the desired loss of expression or activity, by using interfering RNA techniques, or the like, are also well known in the art. See, e.g., the references herein and the following section.

### Disruption of Gene Expression

Gene expression (e.g., transcription and/or translation) can be disrupted using any of a variety of techniques known in the art. For example, gene expression can be reduced or eliminated using an antisense nucleic acid, an interfering RNA, or a microRNA.

Use of antisense nucleic acids is well known in the art. An antisense nucleic acid has a region of complementarity to a target nucleic acid, e.g., a target gene, mRNA, or cDNA. Typically, a nucleic acid comprising a nucleotide sequence in a complementary, antisense orientation with respect to a coding (sense) sequence of an endogenous gene is introduced into a cell. The antisense nucleic acid can be RNA, DNA, a PNA or any other appropriate molecule. A duplex can form between the antisense sequence and its complementary sense sequence, resulting in inactivation of the gene. The antisense nucleic acid can inhibit gene expression by forming a duplex with an RNA transcribed from the gene, by forming a triplex with duplex DNA, etc. An antisense nucleic acid can be produced, e.g., for any gene whose coding sequence is known or can be determined by a number of well-established techniques (e.g., chemical synthesis of an antisense RNA or oligonucleotide (optionally including modified nucleotides and/or linkages that increase resistance to degradation or improve cellular uptake) or in vitro transcription). Antisense nucleic acids and their use are described, e.g., in U.S. Pat. No. 6,242,258 to Haselton and Alexander (Jun. 5, 2001) entitled “Methods for the selective regulation of DNA and RNA transcription and translation by photoactivation”; U.S. Pat. No. 6,500,615; U.S. Pat. No. 6,498,035; U.S. Pat. No. 6,395,544; U.S. Pat. No. 5,563,050; E. Schuch et al (1991) Symp Soc. Exp Biol 45:117-127; de Lange et al., (1995) Curr Top Microbiol Immunol 197:57-75; Hamilton et al. (1995) Curr Top Microbiol Immunol 197:77-89; Finnegan et al., (1996) Proc Natl Acad Sci USA 93:8449-8454; Uhlmann and A. Pepan (1990), Chem. Rev. 90:543; P. D. Cook (1991), Anti-Cancer Drug Design 6:585; J. Goodchild, Bioconjugate Chem. 1 (1990) 165; and, S. L. Beaucage and R. P. Iyer (1993), Tetrahedron 49:6123; and F. Eckstein, Ed. (1991), Oligonucleotides and Analogues—A Practical Approach, IRL Press.

Gene expression can also be inhibited by RNA silencing or interference. In the context of this invention, “RNA silencing” refers to any mechanism through which the presence of a single-stranded or, typically, a double-stranded RNA in a cell results in inhibition of expression of a target gene comprising a sequence identical or nearly identical to that of the RNA, including, but not limited to, RNA interference, repression of translation of a target mRNA transcribed from the target gene without alteration of the mRNA's stability, and transcriptional silencing (e.g., histone acetylation and heterochromatin formation leading to inhibition of transcription of the target mRNA).

The term “RNA interference” (“RNAi,” sometimes called RNA-mediated interference, post-transcriptional gene silencing, or quelling) refers to a phenomenon in which the presence of RNA, typically double-stranded RNA, in a cell results in inhibition of expression of a gene comprising a sequence identical, or nearly identical, to that of the double-stranded RNA. The double-stranded RNA responsible for inducing RNAi is called an “interfering RNA.” Expression of the gene is inhibited by the mechanism of RNAi as described below, in which the presence of the interfering RNA results in degradation of mRNA transcribed from the gene and thus in decreased levels of the mRNA and any encoded protein.

The mechanism of RNAi has been and is being extensively investigated in a number of eukaryotic organisms and cell types. See, for example, the following reviews: McManus and Sharp (2002) “Gene silencing in mammals by small interfering RNAs” Nature Reviews Genetics 3:737-747; Hutvagner and Zamore (2002) “RNAi: Nature abhors a double strand” Curr Opin Genet & Dev 200:225-232; Hannon (2002) “RNA interference” Nature 418:244-251; Agami (2002) “RNAi and related mechanisms and their potential use for therapy” Curr Opin Chem Biol 6:829-834; Tuschl and Borkhardt (2002) “Small interfering RNAs: A revolutionary tool for the analysis of gene function and gene therapy” Molecular Interventions 2:158-167; Nishikura (2001) “A short primer on RNAi: RNA-directed RNA polymerase acts as a key catalyst” Cell 107:415-418; and Zamore (2001) “RNA interference: Listening to the sound of silence” Nature Structural Biology 8:746-750. RNAi is also described in the patent literature; see, e.g., CA 2359180 by Kreutzer and Limmer entitled “Method and medicament for inhibiting the expression of a given gene”; WO 01/68836 by Beach et al. entitled “Methods and compositions for RNA interference”; WO 01/70949 by Graham et al. entitled “Genetic silencing”; and WO 01/75164 by Tuschl et al. entitled “RNA sequence-specific mediators of RNA interference.”

In brief, double-stranded RNA introduced into a cell (e.g., into the cytoplasm) is processed, for example by an RNAse III-like enzyme called Dicer, into shorter double-stranded fragments called small interfering RNAs (siRNAs, also called short interfering RNAs). The length and nature of the siRNAs produced is dependent on the species of the cell, although typically siRNAs are 21-25 nucleotides long (e.g., an siRNA may have a 19 base pair duplex portion with two nucleotide 3′ overhangs at each end). Similar siRNAs can be produced in vitro (e.g., by chemical synthesis or in vitro transcription) and introduced into the cell to induce RNAi. The siRNA becomes associated with an RNA-induced silencing complex (RISC). Separation of the sense and antisense strands of the siRNA, and interaction of the siRNA antisense strand with its target mRNA through complementary base-pairing interactions, optionally occurs. Finally, the mRNA is cleaved and degraded.

Expression of a target gene in a cell can thus be specifically inhibited by introducing an appropriately chosen double-stranded RNA into the cell. Guidelines for design of suitable interfering RNAs are known to those of skill in the art. For example, interfering RNAs are typically designed against exon sequences, rather than introns or untranslated regions. Characteristics of high efficiency interfering RNAs may vary by cell type. For example, although siRNAs may require 3′ overhangs and 5′ phosphates for most efficient induction of RNAi in Drosophila cells, in mammalian cells blunt ended siRNAs and/or RNAs lacking 5′ phosphates can induce RNAi as effectively as siRNAs with 3′ overhangs and/or 5′ phosphates (see, e.g., Czauderna et al. (2003) “Structural variations and stabilizing modifications of synthetic siRNAs in mammalian cells” Nucl Acids Res 31:2705-2716). As another example, since double-stranded RNAs greater than 30-80 base pairs long activate the antiviral interferon response in mammalian cells and result in non-specific silencing, interfering RNAs for use in mammalian cells are typically less than 30 base pairs (for example, Caplen et al. (2001) “Specific inhibition of gene expression by small double-stranded RNAs in invertebrate and vertebrate systems” Proc. Natl. Acad. Sci. USA 98:9742-9747, Elbashir et al. (2001) “Duplexes of 21-nucleotide RNAs mediate RNA interference in cultured mammalian cells” Nature 411:494-498 and Elbashir et al. (2002) “Analysis of gene function in somatic mammalian cells using small interfering RNAs” Methods 26:199-213 describe the use of 21 nucleotide siRNAs to specifically inhibit gene expression in mammalian cell lines, and Kim et al. (2005) “Synthetic dsRNA Dicer substrates enhance RNAi potency and efficacy” Nature Biotechnology 23:222-226 describes use of 25-30 nucleotide duplexes). The sense and antisense strands of a siRNA are typically, but not necessarily, completely complementary to each other over the double-stranded region of the siRNA (excluding any overhangs). The antisense strand is typically completely complementary to the target mRNA over the same region, although some nucleotide substitutions can be tolerated (e.g., a one or two nucleotide mismatch between the antisense strand and the mRNA can still result in RNAi, although at reduced efficiency). The ends of the double-stranded region are typically more tolerant to substitution than the middle; for example, as little as 15 bp (base pairs) of complementarity between the antisense strand and the target mRNA in the context of a 21 mer with a 19 bp double-stranded region has been shown to result in a functional siRNA (see, e.g., Czauderna et al. (2003) “Structural variations and stabilizing modifications of synthetic siRNAs in mammalian cells” Nucl Acids Res 31:2705-2716). Any overhangs can but need not be complementary to the target mRNA; for example, TT (two 2′-deoxythymidines) overhangs are frequently used to reduce synthesis costs.

Although double-stranded RNAs (e.g., double-stranded siRNAs) were initially thought to be required to initiate RNAi, several recent reports indicate that the antisense strand of such siRNAs is sufficient to initiate RNAi. Single-stranded antisense siRNAs can initiate RNAi through the same pathway as double-stranded siRNAs (as evidenced, for example, by the appearance of specific mRNA endonucleolytic cleavage fragments). As for double-stranded interfering RNAs, characteristics of high-efficiency single-stranded siRNAs may vary by cell type (e.g., a 5′ phosphate may be required on the antisense strand for efficient induction of RNAi in some cell types, while a free 5′ hydroxyl is sufficient in other cell types capable of phosphorylating the hydroxyl). See, e.g., Martinez et al. (2002) “Single-stranded antisense siRNAs guide target RNA cleavage in RNAi” Cell 110:563-574; Amarzguioui et al. (2003) “Tolerance for mutations and chemical modifications in a siRNA” Nucl. Acids Res. 31:589-595; Holen et al. (2003) “Similar behavior of single-strand and double-strand siRNAs suggests that they act through a common RNAi pathway” Nucl. Acids Res. 31:2401-2407; and Schwarz et al. (2002) Mol. Cell. 10:537-548.

Due to currently unexplained differences in efficiency between siRNAs corresponding to different regions of a given target mRNA, several siRNAs are typically designed and tested against the target mRNA to determine which siRNA is most effective. Interfering RNAs can also be produced as small hairpin RNAs (shRNAs, also called short hairpin RNAs), which are processed in the cell into siRNA-like molecules that initiate RNAi (see, e.g., Siolas et al. (2005) “Synthetic shRNAs as potent RNAi triggers” Nature Biotechnology 23:227-231). Such hairpins can be encoded by genes introduced into the cell, optionally under the control of inducible or other desired promoters.

Further details on RNAi and induction thereof are available in the art; see, e.g., references herein. For construction of transgenic Drosophila in which RNAi of a given gene is inducible and/or heritable, for example, see also Takemae et al. (2003) “Proteoglycan UDP-Galactose:β-Xylose β1,4-Galactosyltransferase 1 Is Essential for Viability in Drosophila melanogaster” J. Biol. Chem. 278:15571-15578, Kamiyama et al. (2003) “Molecular Cloning and Identification of 3′-Phosphoadenosine 5′-Phosphosulfate Transporter” J. Biol. Chem. 278:25958-25963, Ichimiya et al. (2004) “The Twisted Abdomen Phenotype of Drosophila POMT1 and POMT2 Mutants Coincides with Their Heterophilic Protein O-Mannosyltransferase Activity” J. Biol. Chem. 279:42638-42647, and Kwon et al. (2003) “The Drosophila Selenoprotein BthD Is Required for Survival and Has a Role in Salivary Gland Development” Mol Cell Biol. 23:8495-8504.

The presence of RNA, particularly double-stranded RNA, in a cell can result in inhibition of expression of a gene comprising a sequence identical or nearly identical to that of the RNA through mechanisms other than RNAi. For example, double-stranded RNAs that are partially complementary to a target mRNA can repress translation of the mRNA without affecting its stability. As another example, double-stranded RNAs can induce histone methylation and heterochromatin formation, leading to transcriptional silencing of a gene comprising a sequence identical or nearly identical to that of the RNA (see, e.g., Schramke and Allshire (2003) “Hairpin RNAs and retrotransposon LTRs effect RNAi and chromatin-based gene silencing” Science 301:1069-1074; Kawasaki and Taira (2004) “Induction of DNA methylation and gene silencing by short interfering RNAs in human cells” Nature 431:211-217; and Morris et al. (2004) “Small interfering RNA-induced transcriptional gene silencing in human cells” Science 305:1289-1292).

Short RNAs called microRNAs (miRNAs) have been identified in a variety of species. Typically, these endogenous RNAs are each transcribed as a long RNA and then processed to a pre-miRNA of approximately 60-75 nucleotides that forms an imperfect hairpin (stem-loop) structure. The pre-miRNA is typically then cleaved, e.g., by Dicer, to form the mature miRNA. Mature miRNAs are typically approximately 21-25 nucleotides in length, but can vary, e.g., from about 14 to about 25 or more nucleotides. Some, though not all, miRNAs have been shown to inhibit translation of mRNAs bearing partially complementary sequences. Such miRNAs contain one or more internal mismatches to the corresponding mRNA that are predicted to result in a bulge in the center of the duplex formed by the binding of the miRNA antisense strand to the mRNA (e.g., FIG. 32). The miRNA typically forms approximately 14-17 Watson-Crick base pairs with the mRNA; additional wobble base pairs can also be formed. In addition, short synthetic double-stranded RNAs (e.g., similar to siRNAs) containing central mismatches to the corresponding mRNA have been shown to repress translation (but not initiate degradation) of the mRNA. See, for example, Zeng et al. (2003) “MicroRNAs and small interfering RNAs can inhibit mRNA expression by similar mechanisms” Proc. Natl. Acad. Sci. USA 100:9779-9784; Doench et al. (2003) “siRNAs can function as miRNAs” Genes & Dev. 17:438-442; Bartel and Bartel (2003) “MicroRNAs: At the root of plant development?” Plant Physiology 132:709-717; Schwarz and Zamore (2002) “Why do miRNAs live in the miRNP?” Genes & Dev. 16:1025-1031; Tang et al. (2003) “A biochemical framework for RNA silencing in plants” Genes & Dev. 17:49-63; Meister et al. (2004) “Sequence-specific inhibition of microRNA- and siRNA-induced RNA silencing” RNA 10:544-550; Nelson et al. (2003) “The microRNA world: Small is mighty” Trends Biochem. Sci. 28:534-540; Scacheri et al. (2004) “Short interfering RNAs can induce unexpected and divergent changes in the levels of untargeted proteins in mammalian cells” Proc. Natl. Acad. Sci. USA 101:1892-1897; Sempere et al. (2004) “Expression profiling of mammalian microRNAs uncovers a subset of brain-expressed microRNAs with possible roles in murine and human neuronal differentiation” Genome Biology 5:R13; Dykxhoorn et al. (2003) “Killing the messenger: Short RNAs that silence gene expression” Nature Reviews Molec. and Cell Biol. 4:457-467; McManus (2003) “MicroRNAs and cancer” Semin Cancer Biol. 13:253-288; and Stark et al. (2003) “Identification of Drosophila microRNA targets” PLoS Biol. 1:E60.

The cellular machinery involved in translational repression of mRNAs by partially complementary RNAs (e.g., certain miRNAs) appears to partially overlap that involved in RNAi, although, as noted, translation of the mRNAs, not their stability, is affected and the mRNAs are typically not degraded.

The location and/or size of the bulge(s) formed when the antisense strand of the RNA binds the mRNA can affect the ability of the RNA to repress translation of the mRNA. Similarly, location and/or size of any bulges within the RNA itself can also affect efficiency of translational repression. See, e.g., the references above. Typically, translational repression is most effective when the antisense strand of the RNA is complementary to the 3′ untranslated region (3′ UTR) of the mRNA. Multiple repeats, e.g., tandem repeats, of the sequence complementary to the antisense strand of the RNA can also provide more effective translational repression; for example, some mRNAs that are translationally repressed by endogenous miRNAs contain 7-8 repeats of the miRNA binding sequence at their 3′ UTRs. It is worth noting that translational repression appears to be more dependent on concentration of the RNA than RNA interference does; translational repression is thought to involve binding of a single mRNA by each repressing RNA, while RNAi is thought to involve cleavage of multiple copies of the mRNA by a single siRNA-RISC complex.

Guidance for design of a suitable RNA to repress translation of a given target mRNA can be found in the literature (e.g., the references above and Doench and Sharp (2004) “Specificity of microRNA target selection in translational repression” Genes & Dev. 18:504-511; Rehmsmeier et al. (2004) “Fast and effective prediction of microRNA/target duplexes” RNA 10:1507-1517; Robins et al. (2005) “Incorporating structure to predict microRNA targets” Proc Natl Acad Sci 102:4006-4009; and Mattick and Makunin (2005) “Small regulatory RNAs in mammals” Hum. Mol. Genet. 14:R121-R132, among many others) and herein. However, due to differences in efficiency of translational repression between RNAs of different structure (e.g., bulge size, sequence, and/or location) and RNAs corresponding to different regions of the target mRNA, several RNAs are optionally designed and tested against the target mRNA to determine which is most effective at repressing translation of the target mRNA (preferably, without inducing endonucleolytic cleavage and degradation of the target mRNA).

### Molecular Biological Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2000 and Current Protocols in Molecular Biology, F. M. Ausubel et al., eds., Current Protocols, a joint venture between Greene Publishing Associates, Inc. and John Wiley & Sons, Inc., (supplemented through 2007). Other useful references, e.g. for cell isolation and culture include Freshney (1994) Culture of Animal Cells, a Manual of Basic Technique, third edition, Wiley-Liss, New York and the references cited therein and Atlas and Parks (Eds.) The Handbook of Microbiological Media (1993) CRC Press, Boca Raton, Fla. Methods of making nucleic acids (e.g., by in vitro amplification, purification from cells, or chemical synthesis), methods for manipulating nucleic acids (e.g., site-directed mutagenesis, by restriction enzyme digestion, ligation, etc.), and various vectors, promoters, cell lines and the like useful in manipulating and making nucleic acids and polypeptides are described in the above references. In addition, essentially any polynucleotide can be custom or standard ordered from any of a variety of commercial sources. For a description of the basic paradigm of molecular biology, including the expression (transcription and/or translation) of DNA into RNA into protein, see, Alberts et al. (2002) Molecular Biology of the Cell, 4^{th} Edition Taylor and Francis, Inc., and Lodish et al. (1999) Molecular Cell Biology, 4^{th} Edition W H Freeman & Co.

A variety of protein detection methods are known and can be used to determine protein expression levels. Examples include, but are not limited to, Western blots, dot blots, immunoprecipitation, ELISA, and immunoPCR. Similarly, a large number of assays for detecting activity levels of various enzymes and other proteins have been described.

In addition to the various references noted above, a variety of protein manipulation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles. High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

Proteomic detection methods, which detect many proteins simultaneously, have also been described. These can include various multidimensional electrophoresis methods (e.g., 2-dimensional gel electrophoresis), mass spectrometry based methods (e.g., SELDI, MALDI, electrospray, etc.), or surface plasmon resonance methods. For example, in MALDI, a sample is usually mixed with an appropriate matrix, placed on the surface of a probe and examined by laser desorption/ionization. The technique of MALDI is well known in the art. See, e.g., U.S. Pat. No. 5,045,694 (Beavis et al.), U.S. Pat. No. 5,202,561 (Gleissmann et al.), and U.S. Pat. No. 6,111,251 (Hillenkamp). Similarly, for SELDI, a first aliquot is contacted with a solid support-bound (e.g., substrate-bound) adsorbent. A substrate is typically a probe (e.g., a biochip) that can be positioned in an interrogatable relationship with a gas phase ion spectrometer. SELDI is also a well known technique; see, e.g. Issaq et al. (2003) “SELDI-TOF MS for Diagnostic Proteomics” Analytical Chemistry 75:149A-155A.

Similarly, nucleic acid expression levels (e.g., mRNA) can be detected using any available method, including but not limited to Northern analysis, quantitative RT-PCR, microarray analysis, or the like. References sufficient to guide one of skill through these methods are readily available, including Ausubel, Sambrook and Berger (all supra).

### Sequence Comparison, Identity, and Homology

Of particular interest in the present invention are nucleic acids that encode a protein component of the adenylyl cyclase/cAMP/protein kinase A pathway, NF1 and other genes that regulate the pathway, and polypeptides that are components of or that regulate the pathway. Examples include, but are not limited to, NF1, adenylyl cyclase, protein kinase A, and cAMP phosphodiesterase genes and proteins. As noted above, such genes, nucleic acids, and proteins of interest include those from Drosophila melanogaster as well as homologs and orthologs thereof. Sequences substantially identical to the nucleotide or amino acid sequences thereof are also of interest in the present invention.

The terms “identical” or “percent identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

The phrase “substantially identical,” in the context of two nucleic acids or polypeptides (e.g., DNAs encoding an adenylyl cyclase/cAMP/protein kinase A pathway component, or domain thereof, or the amino acid sequence of an adenylyl cyclase/cAMP/protein kinase A pathway component, or domain thereof) refers to two or more sequences or subsequences that have at least about 60%, about 80%, about 90-95%, about 98%, about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, or over the full length of the two sequences to be compared.

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity (e.g., identity) over 50, 100, 150 or more residues (nucleotides or amino acids) is routinely used to establish homology (e.g., over the full length of the two sequences to be compared). Higher levels of sequence similarity (e.g., identity), e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) are described herein and are generally available. Genes (or proteins) that are homologous are referred to as homologs. Optionally, homologous proteins demonstrate comparable activities (e.g., adenylyl cyclase activity, kinase activity, phosphodiesterase activity, or similar). “Orthologs” are genes (or proteins) in different species that evolved from a common ancestral gene by speciation. Normally, orthologs retain the same or similar function in the course of evolution. As used herein “orthologs” are included in the term “homologs.”

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Life Extension Through Neurofibromin Mitochondrial Regulation and Antioxidant Therapy for Neurofibromatosis-1 in Drosophila Melanogaster

The following sets forth a series of experiments that elucidate the pathophysiology of neurofibromatosis-1 (NF1) in Drosophila melanogaster by inactivation or overexpression of the NF1 gene. NF1 gene mutants had shortened life spans and increased vulnerability to heat and oxidative stress in association with reduced mitochondrial respiration and elevated reactive oxygen species (ROS) production. Flies overexpressing NF1 had increased life spans, improved reproductive fitness, increased resistance to oxidative and heat stress in association with increased mitochondrial respiration and a 60% reduction in ROS production. These phenotypic effects proved to be modulated by the adenylyl cyclase/cyclic AMP (cAMP)/protein kinase A pathway, not the Ras/Raf pathway. Treatment of wild-type D. melanogaster with cAMP analogs increased their life span, and treatment of NF1 mutants with metalloporphyrin catalytic antioxidant compounds restored their life span. Thus, neurofibromin regulates longevity and stress resistance through cAMP regulation of mitochondrial respiration and ROS production, and NF1 can be treated using catalytic antioxidants. These results are also described in Tong et al. (2007) Nature Genetics 39:476-485.

### Phenotype and Pathophysiology of NF1 Mutant Flies

Two homozygous NF1 mutants (designated NF1^{P1} and NF1^{P2}) were generated via P element mutagenesis and tested for their longevity. To control for genetic background effects, the NF1^{P1} and NF1^{P2} strains were backcrossed five generations to the w¹¹¹⁸ (isoCJ1) strain to generate approximately 97% isogenic strains⁷ (Methods).

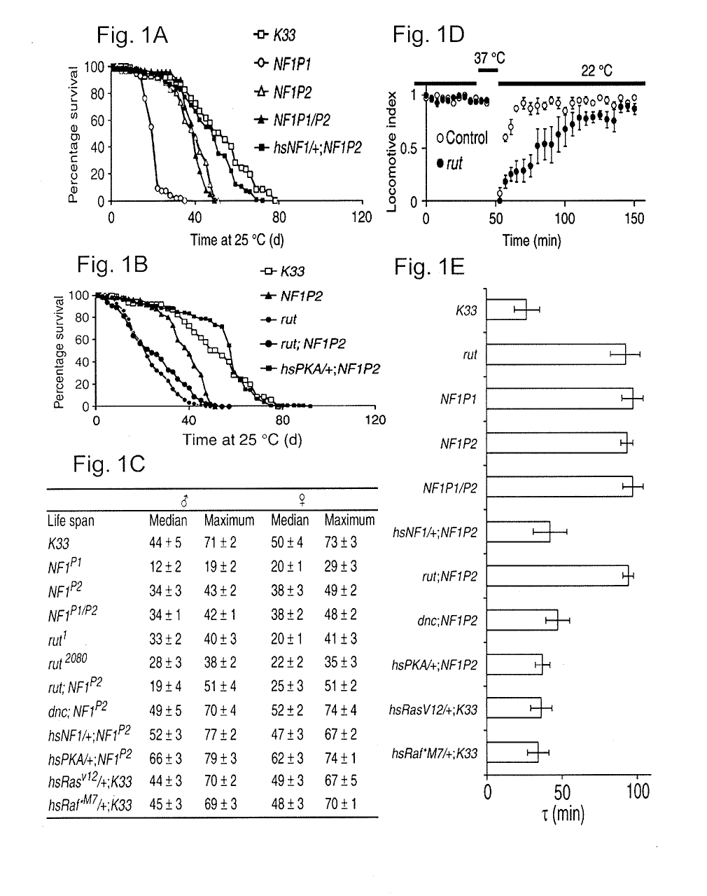

The NF1^{P1} mutant strain had a 60%-73% reduction in life span, and the NF1^{P2} mutant strain had a 24%-40% reduction in life span, relative to the isogenic K33 control strain (FIG. 1 Panel A). The NF1^{P1/P2} intercross heterozygotes had a life span similar to the NF1^{P2} flies (FIG. 1 Panels A-B). The greater life reduction caused by the NF1^{P1} mutation might reflect the disruption of additional functional genes within an intron of NF1. The diminished life span of the NF1^{P2} mutants was rescued by the introduction into these flies of an NF1 transgene (hsNF1) integrated into the 2^{nd} chromosome and driven by the heat shock gene promoter (FIG. 1 Panel A). hsNF1 transgene expression increases in a temperature- and time-dependent manner⁶.

To determine if the reduced life span was due to the upregulation of the Ras/Raf pathway or the downregulation of the adenylyl cyclase/cAMP/PKA pathway, life spans of flies harboring mutations specifically affecting these pathways were tested. The Ras/Raf pathway was upregulated by introducing Ras^{V12} and Raf*^{M7} transgenes, both under heat shock promoter regulation, into flies maintained at 25° C. (refs. 4,5). Both hsRas^{V12}/+; K33 and hsRaf*^{M7}/+; K33 flies had normal life spans (FIG. 1 Panel C). Thus, upregulation of Ras/Raf signaling did not shorten NF1 mutant life spans.

To test if the short life was due to the adenylyl cyclase/cAMP/PKA pathway downregulation, mutant alleles of the neurofibromin-dependent adenylyl cyclase, designated rutabaga (rut)^{6,11}, were analyzed. Two different rutabaga mutants, rut¹ and rut²⁰⁸⁰, had significantly shortened life spans, and the rut and NF1 double mutant (rut; NF1^{P2}) did not further shorten the life span (FIG. 1 Panels B-C), implicating cAMP in mediating longevity. To confirm that the reduced life span in NF1^{P2} was the product of reduced cAMP levels, the NF1^{P2} allele was combined with a cAMP phosphodiesterase gene mutation, dunce (dnc¹), which inhibits cAMP degradation^{10, 11}. The life span of the dnc; NF1^{P2} flies was restored to normal (FIG. 1 Panel C). Normal life was also restored in hsPKA/+; NF1^{P2} flies when NF1^{P2} was combined with the heat shock-inducible and constitutively active PKA (hsPKA*), which upregulates protein phosphorylation⁶ (FIG. 1 Panel B). Therefore, the reduced life span of the NF1 mutants is the product of the impairment of adenylyl cyclase/cAMP/PKA signaling.

To determine the effect of NF1 mutations on D. melanogaster physical fitness, the observation that NF1 mutants showed an inhibited escape response⁸ was exploited. To quantify this phenotype, flies in a vial were startled by tapping them down to the bottom of the vial and their up-climbing capacity was then measured before, during and after a 20-min 37° C. heat stress (FIG. 1 Panel D). The percentage of flies that climbed up 10 mm in 15 s was defined as the locomotive index, and the time necessary for the locomotive index to reach above 90% was defined as the recovery time (τ) (FIG. 1 Panel E). NF1^{P1}, NF1^{P2} and NF1^{P1/P2} flies required over 100 min to recover from the heat stress, compared with 30 min for the K33 control. The reduced heat tolerance of the NF1 mutants was not due to the overexpression of the Ras/Raf pathway, as hsRaf*^{M7}/+; K33 and hsRas^{V12}/+; K33 flies showed T values similar to control K33 flies (FIG. 1 Panel E). On the other hand, rut and rut; NF1^{P2} flies had the same delayed recovery time as the NF1^{P1/P2} mutant flies (FIG. 1 Panel E). Moreover, when NF1^{P2} was combined with hsPKA* (to give hsPKA*; NF1^{P2} flies) or dnc (dnc; NF1^{P2}), τ returned to the normal range (FIG. 1 Panel E). Thus, the reduced tolerance of the NF1 flies to heat stress must also be the product of the adenylyl cyclase/cAMP/PKA pathway downregulation.

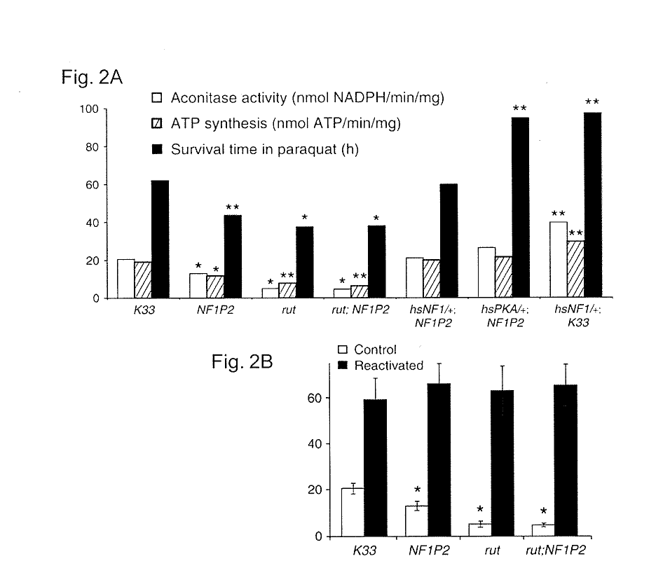

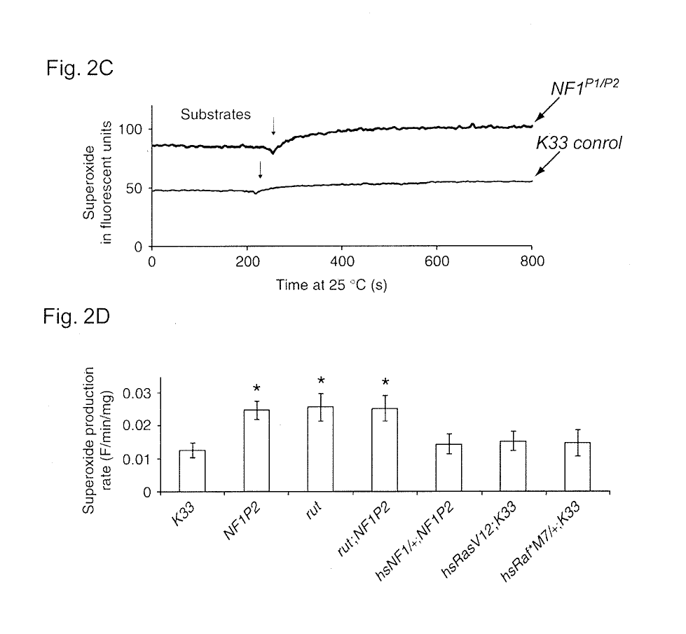

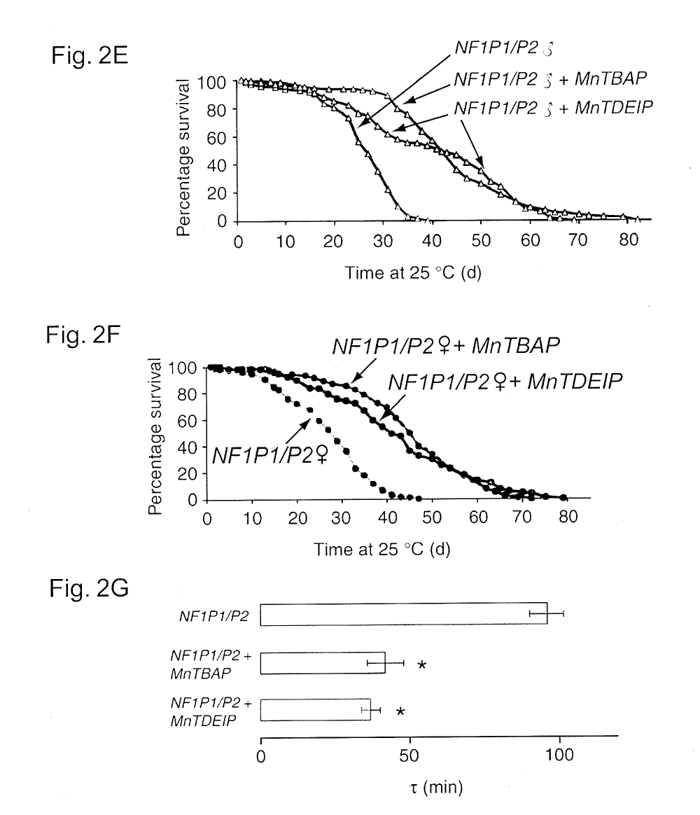

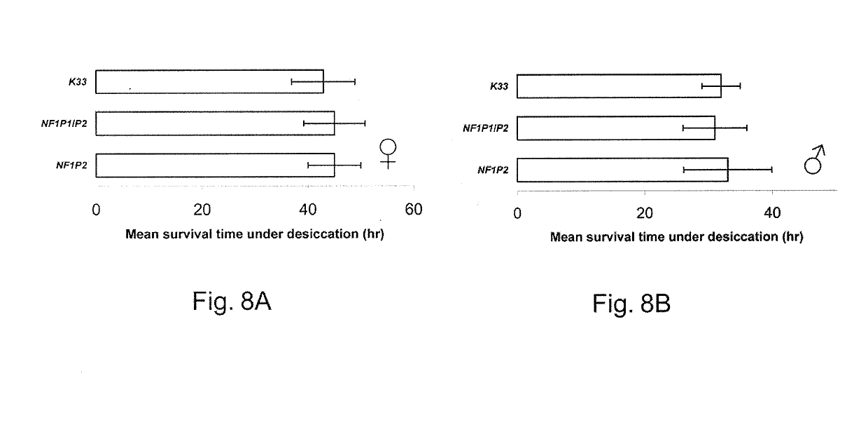

To further investigate the fitness of NF1 mutants, their tolerance to desiccation and oxidative stress was tested. NF1^{P2} flies proved to be as resistant to desiccation as K33 controls (FIG. 8), but NF1^{P2}, rut and rut; NF1^{P2} flies were all significantly more sensitive to paraquat-induced oxidative stress than controls (FIG. 2 Panel A). Paraquat generates intracellular superoxide anion (O₂—) through redox cycling¹². The paraquat sensitivity of NF1^{P2} flies was eliminated in hsNF1/+; NF1^{P2} flies (FIG. 2 Panel A). Furthermore, neurofibromin overexpression on a wild-type background (hsNF1/+; K33) or PKA overexpression on NF1 mutant background (hsPKA*/+; NF1^{P2}) greatly increased resistance to paraquat-induced oxidative stress (FIG. 2 Panel A).

As the mitochondria are a major source of endogenous ROS', and increased ROS could sensitize cells to paraquat, mitochondrial aconitase activity was assayed. The relative specific activity of aconitase is an effective indicator of endogenous mitochondrial oxidative stress, as the aconitase iron-sulfur center is particularly prone to superoxide anion inactivation^{14, 15}. In 30-d-old flies, mitochondrial aconitase activities were reduced by 36% in NF1^{P2} flies, 75% in rut flies and 76% in the double mutant rut; NF1^{P2} flies, relative to age-matched controls (FIG. 2 Panel A). Furthermore, aconitase activity was restored by the hsNF1 transgene (hsNF1; NF 1^{P2}) and was increased more than 90% when neurofibromin was overexpressed on a wild-type background (hsNF1/+; K33) or when the constitutive PKA was overexpressed even in the absence of NF1 (hsPKA*; NF1^{P2}) (FIG. 2 Panel A). Dithiothreitol and iron reactivated the enzymatic activities to the same degree in both mutant and control flies (FIG. 2 Panel B), confirming that aconitase activity reductions were due to the inactivation of existing aconitase instead of reduced expression of the enzyme. Thus, inactivation of the NF1 gene increased oxidative stress, mediated by downregulation of adenylyl cyclase/cAMP/PKA.

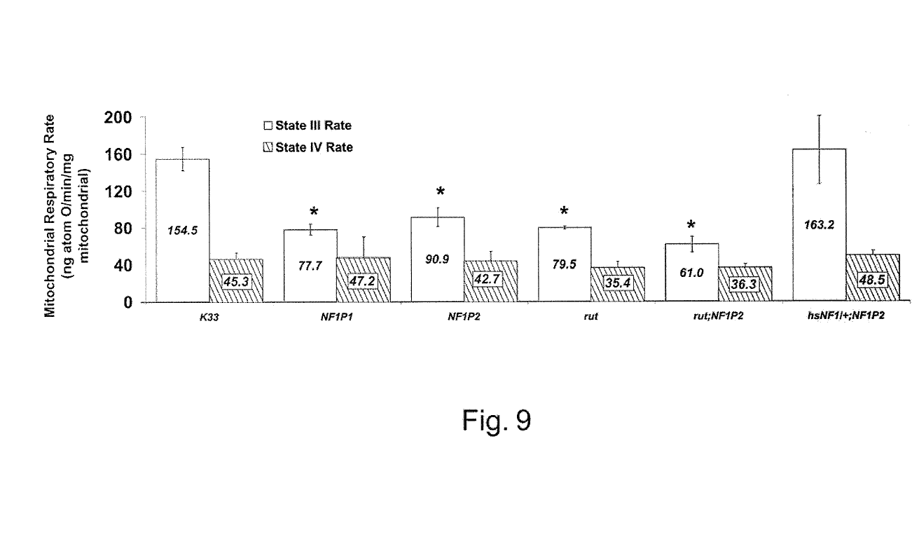

As inhibition of the mitochondrial electron transport chain can increase ROS, respiration rates of NF1 mutant mitochondria versus control mitochondria were analyzed during metabolism of the NADH-linked complex I substrates pyruvate and malate. The ADP-stimulated (state III) respiration rate (FIG. 9) and the derived ATP synthesis rate (FIG. 2 Panel A) were reduced by approximately 50% in NF1^{P1}, NF1^{P2}, rut and rut; NF1^{P2} flies, whereas the non-ADP-stimulated (state IV) respiration was unaffected (FIG. 9). Addition of the heat shock-inducible neurofibromin gene (hsNF1/+; NF1^{P2}) or PKA overexpression (hsPKA*/+; NF1^{P2}) restored the state III respiration rate (FIG. 9) and predicted ATP synthesis rates (FIG. 2 Panel A) to normal levels.

The NF1 mutant mitochondria were then tested for ROS production. Using the MitoSOX fluorescent indicator to monitor superoxide anion production that is a result of the direct transfer of an electron from the electron transport chain to O₂ to generate O₂⁻¹³, NF1^{P1/P2} mitochondria were found to generate more superoxide than control mitochondria (FIG. 2 Panel C). Superoxide levels were increased in rut and rut; NF1^{P2} flies but were reduced to control levels by introduction of the hsNF1 transgene (hsNF1; NF1^{P2}) (FIG. 2 Panel D). Again, overexpression of Ras (hsRas^{V12}/+; K33) or Raf (hsRaf^{M7}/+; K33) did not affect superoxide production (FIG. 2 Panel D), ruling out a role for the Ras/Raf pathway.

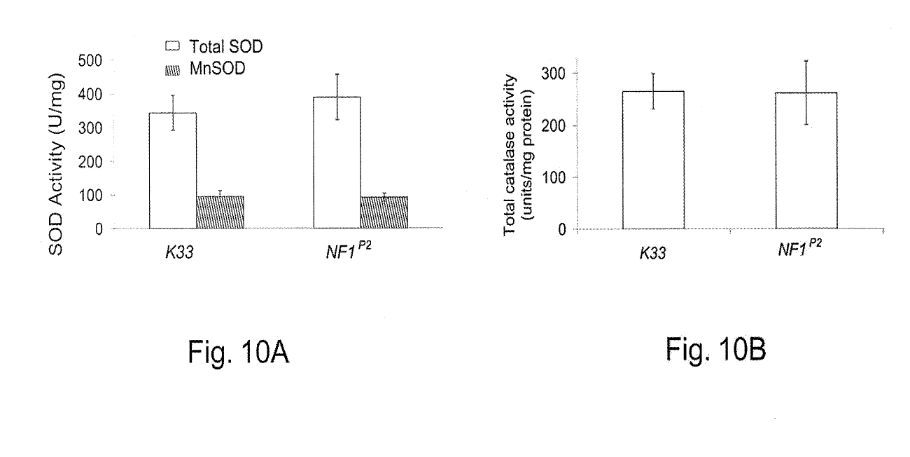

The increased superoxide anion production of NF1^{P1/P2} flies could result from either increased generation of superoxide anion or decreased scavenging by manganese superoxide dismutase (MnSOD). However, analysis of total SOD, MnSOD and catalase activities from NF1^{P2} and K33 flies did not show any significant differences in enzyme activities (FIG. 10). Therefore, the elevated superoxide generated by NF1 mutant flies seems to be the result of increased production, not reduced scavenging.

To further verify that increased superoxide anion production was the cause of the reduced NF1^{P1/P2} life span, NF1^{P1/P2} flies were fed with the metalloporphyrin catalytic antioxidants Mn(III)tetrakis(4-benzoic acid) porphyrin (MnTBAP)¹⁶ and tetrakis(1,3-diethyl imidazolium-2-yl) meso-substituted manganoporphyrin (MnTDEIP)¹⁷, both of which have broad-spectrum antioxidant activities, including SOD activity^{18, 19}. MnTBAP and MnTDEIP exposure increased the survivorship of NF1^{P1/P2} flies by approximately 50% (FIG. 2 Panels E-F). These drugs also enhanced the recovery rate of locomotive performance of NF1^{P1/P2} mutants after heat stress (FIG. 2 Panel G). Therefore, the reduced life span and reduced stress resistance of NF1^{P1/P2} and NF1^{P2} flies, associated with reduced cAMP and mitochondrial respiration, is a direct consequence of increased mitochondrial superoxide anion production.

### Phenotype and Physiology of NF1-Overexpressing Flies

To further validate the importance of the adenylyl cyclase/cAMP/PKA pathway and its effects on mitochondrial respiration and ROS production in regulating life span, stress resistance and fecundity, flies that overexpressed neurofibromin were generated by introducing an extra copy of the NF1 gene, for a total of three copies. Two different strategies were used to overexpress neurofibromin: (i) introduction of the heat shock-inducible NF1 transgene (hsNF1) into wild-type K33 flies and maintenance of the flies at 25° C. and (ii) combining a transgene in which the UAS cis element was fused to the D. melanogaster NF1 gene (UAS-dNF1) with a transgene in which the GAL4 enhancer was driven via either the systemic (Armadillo (Arm))⁸ or neuron-specific (ELAV) promoters (Arm-GAL4 and SLAV-GAL4, respectively).

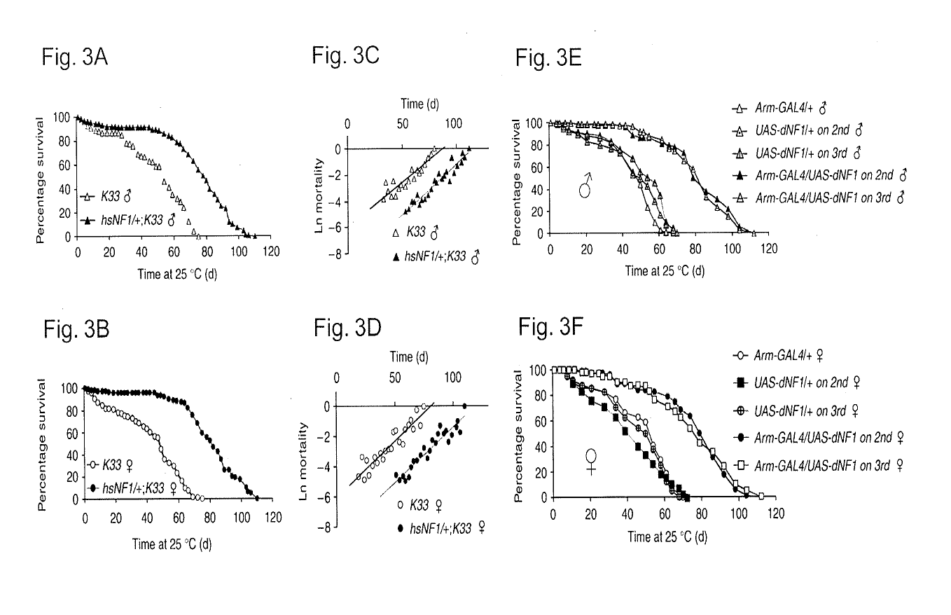

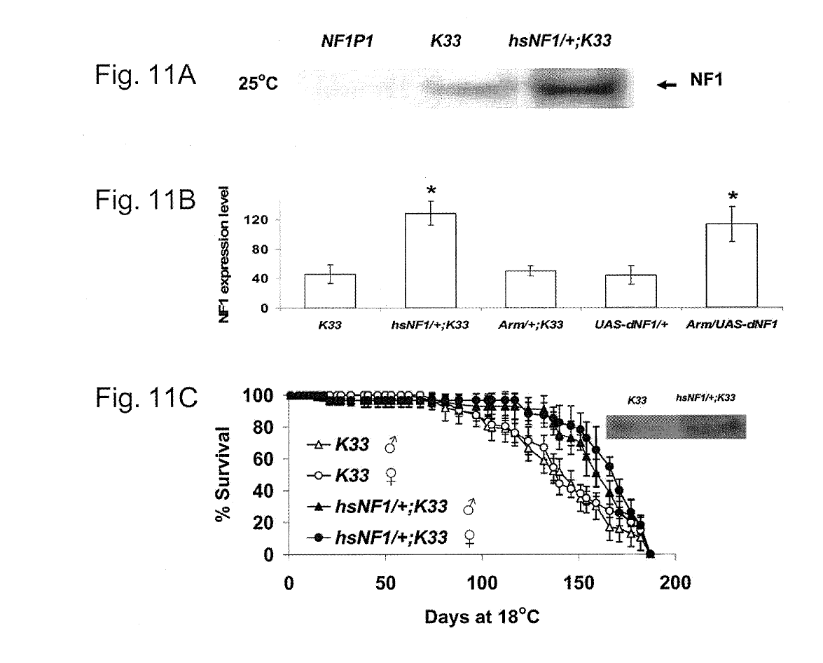

Upregulation of NF1 gene expression by maintaining hsNF1/+; K33 flies at 25° C. throughout life resulted in higher levels of neurofibromin (FIG. 11), an increase in mean life span of 49% for male flies and 68% for females and an increase in maximum life span of 38% for males and 52% for females (FIG. 3 Panels A-B). By contrast, when hsNF1/+; K33 flies were maintained at 25° C. through embryogenesis and 4 d into adulthood but then switched to 18° C. for the remainder of their adult life, the amount of neurofibromin fell to near-control levels, the extension of maximum life span was eliminated and the extension of mean life span was minimized (FIG. 11). The residual increased neurofibromin observed in the 18° C. adults was probably carried over from the prior period when the flies were maintained at 25° C.

Age-specific mortality curves of hsNF1/+; K33 and K33 flies were plotted on a natural log scale (FIG. 3 Panels C-D) and the Gompertz parameters were estimated (Table 1). This showed that the mortality rates of the hsNF1/+; K33 and K33 flies were similar, but their intercepts differed by a factor of 2 to 3. Therefore, neurofibromin overexpression reduces the baseline mortality without altering the age-dependent mortality rate.

TABLE 1
Gompertz mortality parameters were estimated by ln(μₓ) = ln(μ₀) +
ax. The intercept ln(μ₀) is used as an estimate of baseline or
age-independent mortality and the slope a as the rate of
mortality or age-dependent mortality.
Gompertz parameters

| | ♂ | | ♀ | |
|---|---|---|---|---|
| Strain | Intercept | Slope | Intercept | Slope |
| K33^{w} | −5.78 | 0.077 | −6.04 | 0.075 |
| hsNF1/+; K33^{w} | −8.67 | 0.074 | −8.68 | 0.073 |
| Arm-GAL4/+ | −5.03 | 0.072 | −4.45 | 0.077 |
| UAS-dNF1 on 2nd/+ | −3.59 | 0.064 | −3.81 | 0.069 |
| UAS-dNF1 on 3rd/+ | −4.42 | 0.067 | −4.14 | 0.073 |
| Arm-GAL4/UAS-dNF1 2nd | −7.01 | 0.061 | −6.64 | 0.061 |
| Arm-GAL4/UAS-dNF1 3rd | −6.42 | 0.057 | −6.06 | 0.053 |

Neurofibromin was also overexpressed using the GAL4-UAS system⁷. Two independent UAS-dNF1 transgenic lines were studied after outcrossing all transgenic lines five generations to w¹¹¹⁸ to generate an isogenic background. When the UAS-dNF1 lines were crossed with Arm-GAL4 flies to generate double heterozygous animals (Arm-GAL4/+; UAS-dNF1/+), the resulting neurofibromin-overexpressing flies (FIG. 11) showed a mean life span increase of 74% in males and 81% in females and an increase in maximal life span of 65% in males and 70% in females, relative to the homozygous Arm-GAL4 or UAS-dNF1 or heterozygous Arm-GAL4/+ and UAS-dNF1/+ parental strains (FIG. 3 Panels E-F and Table 1). Thus, D. melanogaster life span was extended using two different genetic strategies to increase neurofibromin levels, involving three independent inserts of the NF1 transgene. Therefore, the life span extension must be a product of neurofibromin overexpression and not simply a consequence of heat shock or chromosomal position effects.

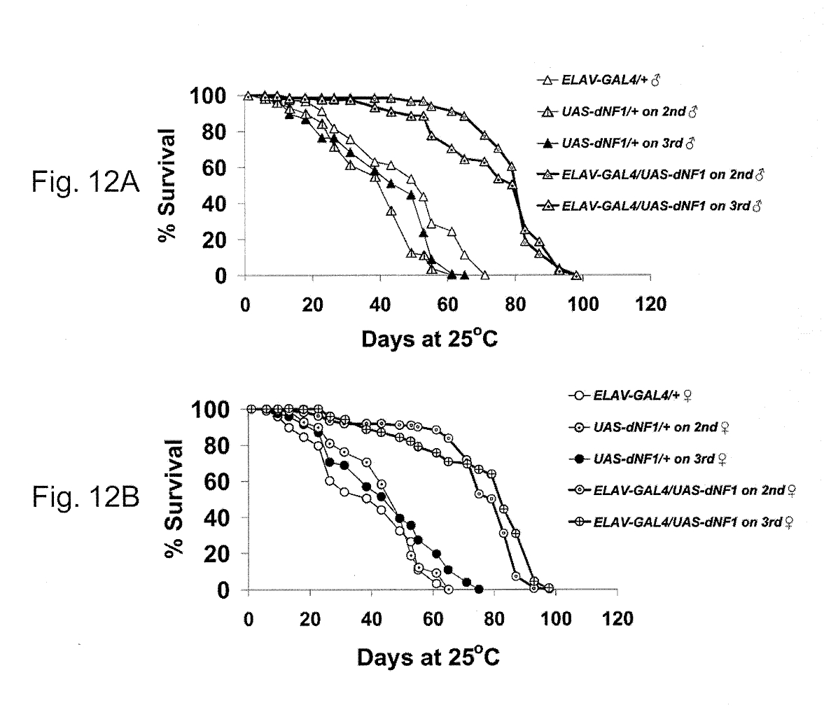

To determine the proportion of the life span extension that was due to neurofibromin protection of neurons^{20, 21}, the UAS-dNF1 transgene was combined with the neuron-specific SLAV promoter (ELAV-GAL4). Both male and female ELAV-GAL4/+; UAS-dNF1/+flies also showed a life span extension of approximately 50% (FIG. 12). Thus, maintaining neuronal integrity must be an important component of longevity.

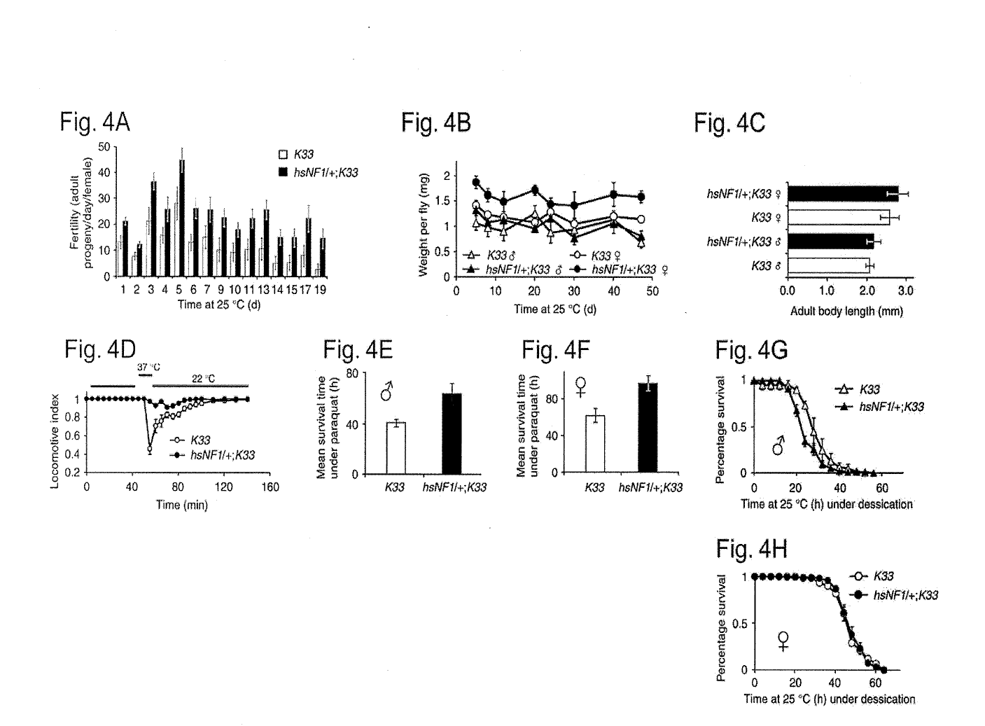

The increased life span of NF1-overexpressing D. melanogaster suggests that they may also be more physically robust. To determine if this was true, the reproductive fertility (FIG. 4 Panel A) and fecundity of the hsNF1/+; K33 females were analyzed. Both fertility and fecundity were higher in the NF1-overexpressing flies than in controls. This was not because the NF1-overexpressing flies were bigger, as both hsNF1/+; K33 males and females had similar body lengths as K33 males and females, respectively (FIG. 4 Panels B-C). Although the hsNF1/+; K33 females were slightly heavier than K33 females (FIG. 4 Panels B-C) this could be because they carried more eggs. Thus, contrary to prevailing evolutionary theories that predict that longevity is inversely correlated with reproductive capacity²², neurofibromin overexpression increased both longevity and fertility.

The stress resistance of NF1-overexpressing flies was tested by assessing up-climbing ability, revealing that the hsNF1/+; K33 flies were highly resistant to heat stress (FIG. 4 Panel D). Similarly, the NF1-overexpressing flies were highly resistant to paraquat-induced oxidative stress, with the hsNF1/+; K33 females showing a 56% increase in survival time and the males a 51% increase in survival time during paraquat exposure, relative to K33 controls (FIG. 4 Panels E-F). However, hsNF1/+; K33 flies were not more resistant to desiccation (FIG. 4 Panels G-H).

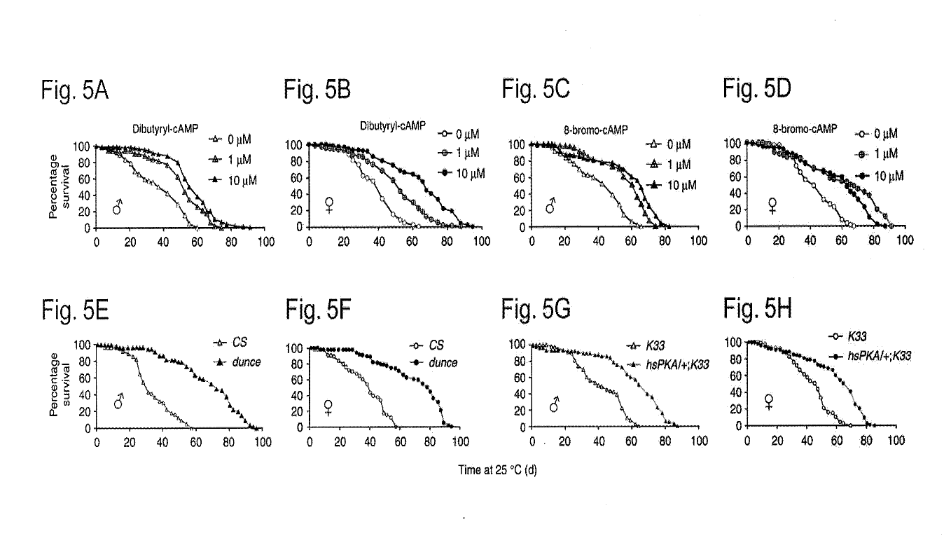

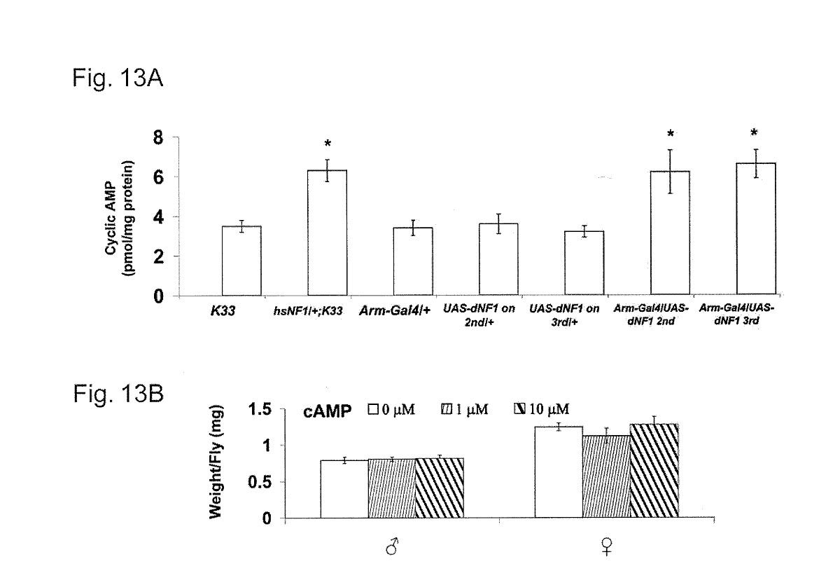

The life span extension of the neurofibromin-overexpressing flies was not due to the downregulation of the Ras/Raf pathway, as flies heterozygous for two different Ras mutants (Ras^{e2F}/+ or Ras^{e1B}/+) (homozygotes being lethal) had normal life spans (data not shown). On the other hand, the life span extension could be accounted for by the upregulation of the adenylyl cyclase/cAMP/PKA pathway. The cAMP concentrations were elevated approximately twofold in NF1-overexpressing hsNF1/+; K33 and Arm-GAL4/+; UAS-dNF/+ strains, relative to K33 or Arm-GAL4/+ of USA-dNF1/+ controls (FIG. 13). Moreover, feeding w¹¹¹⁸ flies the cell-permeable cAMP analogs dibutyryl-cAMP (db-cAMP) and 8-bromo-cAMP increased life span. Adult males that were fed 1 μM or 10 μM db-cAMP showed a 30% increase in median life span, females that were fed 1 μM db-cAMP showed a 60% increase in life span and females fed 10 μM db-cAMP showed a >100% increase in life span (FIG. 5 Panels A-B). Similar findings were obtained by 8-bromo-cAMP feeding (FIG. 5 Panels C-D). This life span extension was unlikely to be due to calorie restriction, as the body weights of both males and females remained the same as controls (FIG. 13).

As a further demonstration of the importance of increased cAMP in life extension, cAMP phosphodiesterase-deficient dunce mutants lived significantly longer than their control Canton S counterparts (FIG. 5 Panels E-F). Similarly, PKA-overexpressing flies (hsPKA*/+flies at 25° C.) also had extended life spans (FIG. 5 Panels G-H). Thus, the increased life span of the NF1-overexpressing flies can be recapitulated by the adenylyl cyclase/cAMP/PKA activation.

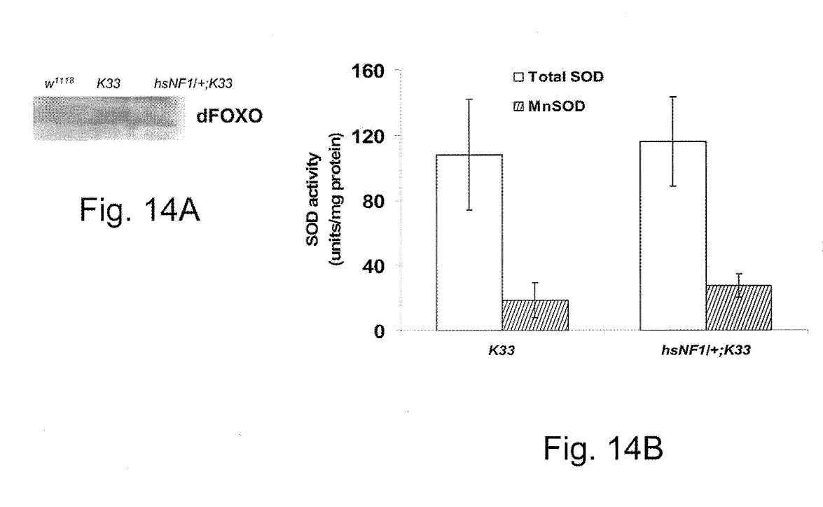

In many systems, life span extension is associated with inactivation of the insulin-like growth factor receptor-protein kinase B (Akt1/2 kinase) pathway. This results in the dephosphorylation and activation of the forkhead transcription factors (FOXO) and induction of MnSOD¹³. However, inactivation of this pathway did not seem to account for the life extension resulting from NF1 overexpression, as no significant differences were found in dephosphorylated FOXO or MnSOD levels between hsNF1/+; K33 and K33 (FIG. 14).

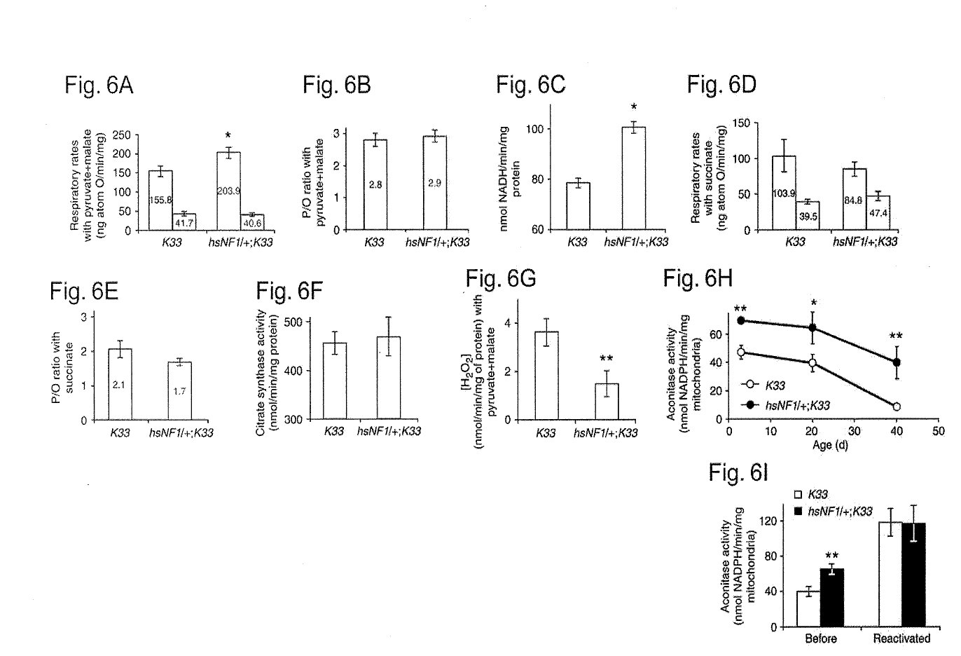

As NF1 inactivation decreased mitochondrial respiration and increased ROS production, increased neurofibromin was predicted to have the opposite effects. Indeed, mitochondrial NADH-linked (pyruvate+malate) respiration rates of mitochondria from NF1-overexpressing flies (hsNF1/+; K33 or Arm-GAL4/+; UAS-dNF1/+) were 25%-38% higher than those of K33 or Arm-GAL4/+; UAS-dNF1/+ control mitochondria, in the presence of ADP (state III respiration), but not in the absence of ADP (state IV respiration) (FIG. 6 Panel A and Table 2). Consequently, the respiratory control ratio (RCR) (state III O₂ consumption rate/state IV O₂ consumption rate) was significantly higher for mitochondria from NF1-overexpressing flies (hsNF1/+; K33) than for K33 mitochondria (4.8 versus 3.4, n=6, P=0.02, t test) (FIG. 6 Panel A) and for Arm-GAL4/UAS-dNF1 mitochondria versus control Arm-GAL4/+ or UAS-dNF1 mitochondria (5.3 versus 3.6, n=5, P=0.008, t test) (Table 2). As the OXPHOS coupling efficiency (P/O ratio) was not changed (FIG. 6 Panel B), the NF1-overexpressing (hsNF1/+; K33) mitochondria are calculated to have a 54% higher ATP production rate, when metabolizing NADH-linked substrates, than K33 control mitochondria (FIG. 2 Panel A), which parallels an increase in complex I activity (FIG. 6 Panel C).

TABLE 2
Overexpression of NF1 using the GAL4/UAS system increases mitochondrial respiration and
complex I activity, reduces ROS production and stabilizes mitochondrial aconitase activity.

| | | UAS- | UAS- | Arm-GAL4/ | Arm-GAL4/ |
|---|---|---|---|---|---|
| | Arm- | dNF1/+ on | dNF1/+ on | UAS-dNF1 | UAS-dNF1 |
| | GAL4/+ | 2nd chr. | 3rd chr. | on 2nd chr. | on 3rd chr. |
| Complex I substrates | | | | | |
| State III | 150.6 | 157.1 | 152.3 | 210.6^{a} | 212.3^{a} |
| State IV | 42.1 | 39.5 | 43.3 | 39.4 | 40.1 |
| (ng atom O/min/mg) | | | | | |
| P/O ratio | 2.9 | 2.8 | 2.9 | 2.9 | 2.7 |
| Complex II substrates | | | | | |
| State III | 105.3 | 110.9 | 101.4 | 103.9 | 106.4 |
| State IV | 44.3 | 43.1 | 39.5 | 40.3 | 41.8 |
| (ng atom O/min/mg) | | | | | |
| Complex I activity | 74.4 | 75.2 | 78.5 | 110.1^{b} | 107.6^{b} |
| (nmol NADH/min/mg | | | | | |
| protein) | | | | | |
| Citrate cynthase | 459.1 | 463.8 | 467.0 | 470.9 | 468.2 |
| (nmol/min/mg protein) | | | | | |
| H₂O₂ | 3.2 | 3.5 | 3.1 | 1.4^{b} | 1.3^{b} |
| (nmol/min/mg protein) | | | | | |
| Aconitase | 33.1 | 39.7 | 42.5 | 70.8^{a} | 67.6^{a} |
| (nmol NADPH/min/mg) | | | | | |

Complex I (pyruvate + malate) and complex II (succinate) respiration rates, P/O ratio and complex I activity measurements were measured on mitochondria isolated from pooling 100 5-d-old flies (50 males and 50 females).
The mean of five experiments are shown.
Citrate synthase activities are averages of three independent experiments.
H₂O₂ production rates are averages of three independent mitochondrial isolations from 10-d-old flies.
The aconitase activities are averages of three independent mitochondria isolation from 20-d-old flies.
Chr., chromosome.
^{a}P < 0.05;
^{b}P < 0.01 (t test).

By contrast, the respiration rates recorded when using the FADH₂-linked complex II substrate succinate were essentially the same for neurofibromin-overexpressing mitochondria (hsNF1/+; K33) versus control K33 mitochondria (FIG. 6 Panel D) and for Arm-GAL4/UAS-dNF1 versus Arm-GAL4/+ or UAS-dNF1 mitochondria (Table 2), with or without ADP. Thus, the succinate-based RCR for hsNF1/+; K33 versus K33 mitochondria (1.9 versus 2.3 (n=4, P=0.26, t test)) and for Arm-GAL4/UAS-dNF1 versus Arm-GAL4/+ or UAS-dNF1 mitochondria (2.6 versus 2.5 (P=0.49, t test)) were not significantly different, and the P/0 ratios were also unchanged (FIG. 6 Panel E and Table 2).

The NADH-linked and FADH₂-linked respiration pathways share the downstream respiratory complexes III, IV and V, differing only in the initial enzyme complex (complex I for the NADH-linked substrates and complex II for succinate). Thus, the differences in respiration rate observed between NF1-overexpressing and control mitochondria are most likely to reside with complex I. This proved to be the case, as complex I activity was 28% higher in NF1-overexpressing hsNF1/+; K33 versus K33 mitochondria (FIG. 6 Panel C) and 38% higher in Arm-GAL4/UAS-dNF1 versus Arm-GAL4/+ or UAS-dNF1 mitochondria (Table 2). Mitochondrial citrate synthase specific activity did not differ between NF1-overexpressing and control mitochondria (FIG. 6 Panel F and Table 2). Thus, the increased cAMP levels associated with increased neurofibromin resulted in increased mitochondrial complex I activity.

Increased neurofibromin levels could also be expected to result in reduced mitochondrial ROS production. As expected, mitochondrial H₂O₂ production was reduced 58%-59% for both hsNF1/+; K33 versus K33 flies (FIG. 6 Panel G) and Arm-GAL4/UAS-dNF1 versus Arm-GAL4/+ or UAS-dNF1 flies (Table 2). This reduced mitochondrial ROS was also associated with increased mitochondrial aconitase activity throughout life, as demonstrated in hsNF1/+; K33 flies at 5, 20, and 40 d of adult age (FIG. 6 Panel H) and also for Arm-GAL4/UAS-dNF1 fly mitochondria (Table 2). These differences in mitochondrial aconitase-specific activity were due to ROS modulation of the enzyme-specific activity, as both NF1-overexpressing and control fly mitochondrial aconitase activities were reactivated up to the same level by treatment with dithiothreitol plus iron (FIG. 6 Panel I). Therefore, the increased longevity associated with NF1 overexpression is mediated by increased cAMP, resulting in increased NADH-linked respiration and complex I activity and decreased mitochondrial ROS production and oxidative stress.

### NF1, AC/cAMP/PKA Pathway, and Life Span

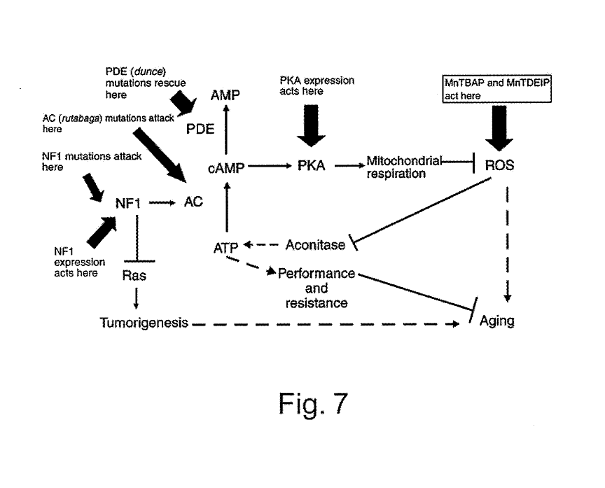

The results herein demonstrate that in D. melanogaster, mutational loss of NF1 reduces life span and stress tolerance, inhibits mitochondrial respiration, and leads to increased mitochondrial ROS production. Conversely, increased NF1 expression markedly extends life span, significantly improves oxidative stress resistance and reproductive fitness, activates mitochondrial complex I, and reduces mitochondrial ROS production and oxidative damage. Moreover, treatment of D. melanogaster with exogenous cAMP markedly increases longevity, whereas treatment of NF1 mutant D. melanogaster with catalytic antioxidants ameliorates the associated reduction in life span. Therefore, at least in D. melanogaster, a significant component of the reduced life span and the other adverse effects of neurofibromin deficiency are mediated by cAMP regulation of mitochondrial function (FIG. 7).

The importance of cellular and mitochondrial oxidative stress in modulating D. melanogaster longevity has been actively investigated. D. melanogaster life span extension has been reported in transgenic flies that systemically overexpress MnSOD and copper/zinc super oxide dismutase (Cu/ZnSOD), the latter either alone or with catalase throughout life^{23, 24, 25}. D. melanogaster life span has also been extended by overexpression of Cu/ZnSOD or MnSOD in motor neurons^{20, 21}. Moreover, flies with reduced life span due to Cu/ZnSOD and MnSOD deficiency that are treated with the catalytic antioxidant EUK-8 (manganese N,N′-bis(salicyidene) ethylenediamine chloride) or with mitochondrially targeted coenzyme Q (MitoQ) partially restored normal life span, although treatment of wild-type flies with EUK-8 or MitoQ shortened their life spans²⁶. The data herein show that treatment of short-lived NF1 mutant flies with MnTBAP restores normal life span, and MnTBAP has been shown to reduce mitochondrial ROS production and oxidative damage^{27,28}. Moreover, NF1 overexpression extends life span in association with reduced mitochondrial ROS generation. Taken together, these results support a central role for mitochondrial ROS in determining the life span of D. melanogaster but also indicate that optimal benefits of antioxidant treatment can be achieved by defining not only the specific ROS to be removed (superoxide anion, hydrogen peroxide, hydroxyl radical), but also the time during development, the cell type and the subcellular compartment to be targeted for ROS reduction.

In multiple animal systems, life span extension has been achieved by the inactivation of the insulin-like receptor signal transduction pathway. When activated by insulin-like ligands, this pathway activates the Akt kinase that phosphorylates and inactivates the forkhead transcription factors (FOXOs). Active forkhead transcription factors upregulate MnSOD and the peroxisome proliferator-activated receptor γcoactivator (PGC-1α) gene. The promoter of the mammalian PGC-1α gene encompasses three insulin response elements (IREs) that bind dephosphorylated and deacetylated FOXO transcription factors and one cAMP response element (CRE) that binds to phosphorylated CREB^{13, 29}. The PGC-1α protein, in turn, interacts with multiple transcription factors to upregulate mitochondrial biogenesis. Although inactivation of the insulin-like growth factor receptor pathway should upregulate mitochondrial biogenesis and reduce mitochondrial ROS production¹³, this is unlikely to explain the modulation of life spans in the studies reported herein, as MnSOD expression and FOXO phosphorylation did not change in NF1 mutant and NF1-overexpressing flies.

However, clear evidence for linkage between cAMP levels and the regulation of life span, mitochondrial respiration and mitochondrial ROS production was found in the experiments described herein. Without limitation to any particular mechanism, according to existing literature, there are two possible mechanisms by which cAMP levels could regulate mitochondrial oxidative phosphorylation: (i) transcriptional regulation of nuclear DNA-encoded mitochondrial genes and/or (ii) direct cAMP-activated PICA modification of complex I polypeptides. In mammalian systems, cAMP upregulates the expression of nuclear DNA-encoded mitochondrial genes through the activation of the PGC-1α gene, presumably through PICA phosphorylation of CREB and the binding of phosphorylated CREB to the CRE element in the PGC-1α gene promoter. This pathway may be a factor in the observations herein, as a PGC-1α homolog has recently been identified in D. melanogaster that induces upregulation of mitochondrial biogenesis through FOXO transcription factors³⁰, and in silico analysis of the upstream sequences of the D. melanogaster PGC-1α gene (CG9809) showed putative cAMP response elements.

Alternatively (or additionally), cAMP can regulate mitochondrial respiration and ROS production by the direct activation of mitochondrial PKA and the phosphorylation of mitochondrial proteins. Mitochondrially localized cAMP-activated PKA has been reported to mediate phosphorylation of the complex I subunits³¹ ESSS and MWFE³² and/or 42 kDa and B14.5A³³. This has been associated with increased complex I Vₘₐₓ, increased NADH⁺-linked but not FADH₂-linked respiration and suppression of mitochondrial ROS production without major alterations in the mitochondrial or cellular antioxidant defense systems^{31, 34, 35, 36}. These observations parallel the present findings in NF1 mutant and NF1-overexpressing D. melanogaster, suggesting that neurofibromin may regulate mitochondrial respiration and ROS production through direct cAMP and PICA-mediated phosphorylation of mitochondrial proteins.

Regardless of the molecular mechanism or the relative importance of nuclear oxidative phosphorylation gene regulation and mitochondrial protein modulation by neurofibromin regulation of cAMP levels, the neurofibromin modulation of mitochondrial respiration and ROS production via cAMP signaling directly implicate the mitochondria in the pathophysiology of NF1. Consistent with this possibility, neurofibromas from individuals with NF1 have been found to acquire somatic mutations in the mtDNA control region³⁷. Both germline and somatic mtDNA mutations have been observed in prostate cancers³⁸, and somatic mtDNA mutations have been observed in a wide range of human tumors³⁹. Moreover, mtDNA mutations have been linked to prostate cancer and associated with increased mitochondrial ROS production³⁸, and ROS has been shown to act as a potent mitogen⁴⁰. Therefore, the increased mitochondrial ROS production resulting from NF1 mutations can be an important factor in the generation of neurofibromas. Suppression of mitochondrial ROS production through the treatment with catalytic antioxidants such as MnTBAP and MnTDEIP can provide a powerful new approach to treat NF1, as well as other cancers.

### Methods

### Fly Stocks

All fly stocks were raised at 25° C. and 40%-50% humidity. K33 is the parental line upon which the NF1 mutant and transgenic lines were generated and thus was used as the control. Strains were compared on the w¹¹¹⁸ (isoCJ1) isogenic background after backcrossing for five generations, yielding a 97% genetic similarity in the flies. Heterozygous hsNF1/+; NF1^{P2}, hsPKA/+; NF1^{P2}, hsRaf*^{M7}/+; K33, hsRas^{V12}/+; K33 and hsNF1/+; K33 flies were studied to avoid any recessive effects resulting from the insertion site of the transgenes. The hsPKA; K33 flies harbor a mouse PKA transgene, with His87Gln and Trp196Arg substitutions that prevent interaction with the PKA regulatory subunit^{6,7}. GAL4 and UAS-D. melanogaster NF1 lines were also backcrossed to w¹¹¹⁸ background for five generations.

### Longevity Assay

Life spans were determined at 25° C. and 50% humidity with a 12-h light/dark cycle. Male and female flies were collected under brief CO₂ anesthesia 2 to 3 d after eclosion, allowing time for mating. The number of deceased flies was recorded every 2 to 3 d, when flies were transferred to fresh cornmeal agar medium. Both Statview 5.0 and Prism GraphPad software were used for survival data and mortality curves analysis. Survival data were analyzed by Kaplan-Meier analysis in Statview 5.0. The log-rank (Mantel-Cox) test results are presented. Censored data were recorded and analyzed with GraphPad software.

### Reproductivity

Fecundity was defined as cumulative number of eggs laid per fertilized female. Fertility was defined as the cumulative number of adult progeny per fertilized female. Each experiment consisted of five females and five males, mated in vials containing standard food and transferred daily at 25° C. The number of eggs laid in each vial were counted as a measure of fecundity, and the vials were kept until eclosion of all the adult progeny to determine fertility. The t test was employed for data analysis.

### Stress Resistance

Stress assays were performed on 2- to 3-d-old flies collected overnight, with 40 flies per vial kept on regular food medium. Paraquat toxicity was tested by starving flies in empty vials for 6 h at 25° C. and then placing them in the vial with a filter paper saturated with 20 mM paraquat and 5% sucrose in distilled water. The effect of desiccation was determined by placing the flies in empty vials at 25° C. In both cases, dead flies were counted every 2 h.

### Physical Activity

Locomotive assays were performed on 3- to 4-d-old flies. Flies were placed in 90 mm×20 mm vertical tubes containing a small quantity of cornmeal food at the bottom with a line drawn horizontally 10 mm from the base. Every 5 min, the vials were tapped until the flies were at the bottom of the vials. Then, flies were given 15 s to climb (up-climbing behavior) toward the top of the vial, and the numbers of flies above the 10 mm line were recorded. The locomotive index is the percentage of flies that climbed up after 15 s. After 30 min of baseline recording at 22° C., flies were subjected to a brief 20-min heat treatment at 37° C. and were then returned to 22° C. for recovery. The locomotive index was then plotted against time over the course of the experiment to determine the recovery time (τ).

cAMP Feeding

cAMP-supplemented food was prepared using dibutyryl-cAMP and 8-bromo-cAMP (Sigma) dissolved in distilled water to prepare a stock solution. The stock solutions were subsequently diluted into food to make the specific concentrations of 1 μM and 10 μM. Red food dye (six drops per 500 ml food) was added to the supplemented food to insure homogeneity. Significant differences were not observed in the weekly measurements of body weight and length in flies fed cAMP analogs compared with control flies, confirming that the cAMP-fed flies did not live longer simply because of drug-induced dietary restriction.

cAMP Concentration Measurement

caMP concentrations were determined using a competitive immunoassay (Assay Designs' Correlate-EIA cAMP kit).

### Mitochondrial Respiration

Mitochondria were isolated by gently crushing 40 to 80 flies in a 10-ml Kontes homogenizer with seven strokes of the pestle in 3 ml homogenization buffer consisting of 225 mM mannitol, 75 mM sucrose, 10 mM MOPS, 1 mM EGTA and 0.5% BSA (pH 7.2) at 4° C. (ref. 38). The extracts were filtered through eight layers of cheesecloth and then centrifuged at 300 g for 3 min in a Beckman Avanti J25. The supernatant was centrifuged at 6,000 g for 10 min to obtain a mitochondrial pellet. Mitochondrial protein was determined by the Bradford method using Bio-Rad reagents and correcting for the BSA content in the homogenization buffer. Respiration rates were determined by oxygen consumption using a Clark-type electrode and metabolic chamber containing 650 μl of reaction buffer consisting of 225 mM mannitol, 75 mM sucrose, 10 mM KCl, 10 mM Tris-HCl and 5 mM KH₂PO₄ (pH 7.2) at 25° C. Mitochondrial ATP production rates were calculated from ADP consumption rates during state III respiration. Experiments used 5-d-old flies except unless otherwise indicated in the text.

### Complex I Activity

The specific activity of complex I (NADH-ubiquinone oxidoreductase) was determined as the rotenone (4 μM)-sensitive NADH oxidation at 340 nm, using the coenzyme Q analog 2,3-dimethyl-5-methyl 6-n-decyl-1,4-benzomethyluinone (DB) as an electron acceptor⁴¹.

### Citrate Synthase

Citrate synthase was analyzed by the reduction of 5,5′-dithiobis-2-nitrobenzoic acid at 412 nm in the presence of acetyl-CoA and oxaloacetate^{4l}.

### ROS Production

H₂O₂ leakage from intact mitochondria respiring on pyruvate and malate was quantified by the horseradish peroxidase-dependent oxidation of p-hydroxyphenol acetic acid. 320 nm excitation and 400 nm emission were monitored via a Perkin Elmer L20B luminescence spectrometer. Hydrogen peroxide levels were interpolated from standard curves. The rate of superoxide anion production was assayed in isolated mitochondria using MitoSOX (Invitrogen) fluorescence at 510 nm excitation/580 nm emission.

### Aconitase Activity and Reactivation

Mitochondrial aconitase activity was measured on mitochondria sonicated four times for 15 s in a Branson 450 sonicator in 50 mM Tris, 30 mM sodium citrate, 0.5 mM MnCl₂ and 0.2 mM NADP (pH 7.3). The conversion of citrate into α-ketoglutarate was monitored at 340 nm at 25° C. using the coupled reduction of NADP to NADPH by 2 units/ml of isocitrate dehydrogenase in 50 mM Tris, 1 mM cysteine, 1 mM sodium citrate and 0.5 mM MnCl₂ (pH 7.4). Aconitase was reactivated by incubation with 2 mM dithiothreitol and 0.2 mM ferrous ammonium sulfate for 5 min before repeating the enzymatic activity assay⁴².

### Superoxide Dismutase and Catalase Activities

Superoxide dismutase activity was measured spectrophotometrically at 560 nm by recording the reduction of nitro blue tetrazolium. One unit of SOD activity was defined as the amount that inhibited nitro blue tetrazolium reduction half-maximally in a 1 ml reaction volume. MnSOD activity was measured by recording the cyanide-inhabitable reduction of nitro blue tetrazolium⁴³. Catalase activity was measured by spectrophotometrically monitoring the change in ultraviolet absorbance at 240 nm after adding whole cell extract or mitochondria alone⁴⁴.

### Antioxidant Feeding

MnTBAP (Oxis) and MnTDEIP (Aeolus Pharmaceuticals) were dissolved in PBS (Mediatech) before further dilution with cornmeal food to make desired concentration. Red food dye (six drops per 500 ml food) was added to the food to ensure homogeneity. Based on the body weight and length of the antioxidant-fed versus control flies monitored weekly, no evidence was observed that suggested differential food intake by the experimental flies.

### Neurofibromin Detection and Quantification

Neurobromin levels were determined by protein blot analysis. Ten flies (five males and five females) of each category were homogenized in fly homogenization buffer containing 60 mM Tris-HCl (pH 6.8), 10% glycerol, 3% SDS, 2-mercaptoethanol, protease inhibitor cocktail (Roche) and phenylmethylsulfonyl fluoride. Protein concentrations were determined by the Bradford method. 40 μg protein per lane was separated by 10% SDS-PAGE (Invitrogen) and then transferred it overnight (15 h) at 200 mA to a nitrocellulose membrane. Nitrocellulose membranes were incubated overnight at 4° C. with antisera to neurofibromin (Bethyl, 1:1,000 dilution). Blots were washed four times with washing buffer (10 min each) and then incubated with horseradish peroxidase-labeled goat antibody against rabbit IgG (Amersham) for 1.5 h at room temperature. Blots were washed four times with washing buffer (5 min each wash), incubated using the ECL protein blotting analysis system (Amersham) for 1 min and exposed to X-ray film for 2 min.

### FOXO Expression

The level of unphosphorylated (and thus active) FOXO transcription factor was evaluated by reacting the above protein blots with a FOXO1 antibody specific for the unphosphorylated form purchased from Cell Signaling. The antibody was diluted 1:1,000.

### REFERENCES

1. Korf, B. R. Malignancy in neurofibromatosis type 1. Oncologist 5, 477-485 (2000).
2. Friedman, J. M. Epidemiology of neurofibromatosis type 1. Am. J. Med. Genet. 89, 1-6 (1999).
3. Arun, D. & Gutmann, D. H. Recent advances in neurofibromatosis type 1. Curr. Opin. Neurol. 17, 101-105 (2004).
4. Williams, J. A., Su, H. S., Bernards, A., Field, J. & Sehgal, A. A circadian output in Drosophila mediated by neurofibromatosis-1 and Ras/MAPK. Science 293, 2251-2256 (2001).
5. Harrisingh, M. C. & Lloyd, A. C. Ras/Raf/ERK signalling and NFL Cell Cycle 3, 1255-1258 (2004).
6. Guo, H. F., Tong, J., Hannan, F., Luo, L. & Zhong, Y. A neurofibromatosis-1-regulated pathway is required for learning in Drosophila. Nature 403, 895-898 (2000).
7. Tong, J., Hannan, F., Zhu, Y., Bernards, A. & Zhong, Y. Neurofibromin regulates G protein-stimulated adenylyl cyclase activity. Nat. Neurosci. 5, 95-96 (2002).
8. The, I. et al. Rescue of a Drosophila NF1 mutant phenotype by protein kinase A. Science 276, 791-794 (1997).
9. Yohay, K. H. The genetic and molecular pathogenesis of NF1 and NF2. Semin. Pediatr. Neurol. 13, 21-26 (2006).
10. Guo, H. F., The, I., Hannan, F., Bernards, A. & Zhong, Y. Requirement of Drosophila NF1 for activation of adenylyl cyclase by PACAP38-like neuropeptides. Science 276, 795-798 (1997).
11. Zhong, Y., Budnik, V. & Wu, C. F. Synaptic plasticity in Drosophila memory and hyperexcitable mutants: role of cAMP cascade. J. Neurosci. 12, 644-651 (1992).
12. Bus, J. S. & Gibson, J. E. Paraquat: model for oxidant-initiated toxicity. Environ. Health Perspect. 55, 37-46 (1984).
13. Wallace, D. C. A mitochondrial paradigm of metabolic and degenerative diseases, aging, and cancer: a dawn for evolutionary medicine. Annu. Rev. Genet. 39, 359-407 (2005).
14. Das, N., Levine, R. L., Orr, W. C. & Sohal, R. S. Selectivity of protein oxidative damage during aging in Drosophila melanogaster. Biochem. J. 360, 209-216 (2001).
15. Yan, L. J., Levine, R. L. & Sohal, R. S. Oxidative damage during aging targets mitochondrial aconitase. Proc. Natl. Acad. Sci. USA [published erratum appears in Proc. Natl. Acad. Sci. USA 1998 Feb. 17; 95(4):1968] 94, 11168-11172 (1997).
16. Day, B. J., Shawen, S., Liochev, S. I. & Crapo, J. D. A metalloporphyrin superoxide dismutase mimetic protects against paraquat-induced endothelial cell injury, in vitro. J. Pharmacol. Exp. Ther. 275, 1227-1232 (1995).
17. Kachadourian, R., Johnson, C. A., Min, E., Spasojevic, I. & Day, B. J. Flavin-dependent antioxidant properties of a new series of meso-N,N′-dialkyl-imidazolium substituted manganese(III) porphyrins. Biochem. Pharmacol. 67, 77-85 (2004).
18. Patel, M. & Day, B. J. Metalloporphyrin class of therapeutic catalytic antioxidants. Trends Pharmacol. Sci. 20, 359-364 (1999).
19. Day, B. J. Catalytic antioxidants: a radical approach to new therapeutics. Drug Discov. Today 9, 557-566 (2004).
20. Parkes, T. L. et al. Extension of Drosophila life span by overexpression of human SOD1 in motorneurons. Nat. Genet. 19, 171-174 (1998).
21. Phillips, J. P., Parkes, T. L. & Hilliker, A. J. Targeted neuronal gene expression and longevity in Drosophila. Exp. Gerontol. 35, 1157-1164 (2000).
22. Williams, G. C. Pleiotropy, natural selection, and the evoluton of senescence. Evolution Int. J. Org. Evolution 11, 398-411 (1957).
23. Orr, W. C. & Sohal, R. S. Extension of life-span by overexpression of superoxide dismutase and catalase in Drosophila melanogaster. Science 263, 1128-1130 (1994).
24. Sun, J., Folk, D., Bradley, T. J. & Tower, J. Induced overexpression of mitochondrial Mn-superoxide dismutase extends the life span of adult Drosophila melanogaster. Genetics 161, 661-672 (2002).
25. Sun, J., Molitor, J. & Tower, J. Effects of simultaneous over-expression of Cu/ZnSOD and MnSOD on Drosophila melanogaster life span. Mech. Ageing Dev. 125, 341-349 (2004).
26. Magwere, T. et al. The effects of exogenous antioxidants on life span and oxidative stress resistance in Drosophila melanogaster. Mech. Ageing Dev. 127, 356-370 (2006).
27. Li, Q. V., Pedersen, C., Day, B. J. & Patel, M. Dependence of excitotoxic neurodegeneration on mitochondrial aconitase inactivation. J. Neurochem. 78, 746-755 (2001).
28. Velsor, L. W. et al. Mitochondrial oxidative stress in human hepatoma cells exposed to stavudine. Toxicol. Appl. Pharmacol. 199, 10-19 (2004).
29. Daitoku, H., Yamagata, K., Matsuzaki, H., Hatta, M. & Fukamizu, A. Regulation of PGC-1 promoter activity by protein kinase B and the forkhead transcription factor FKHR. Diabetes 52, 642-649 (2003).
30. Gershman, B. et al. High resolution dynamics of the transcriptional response to nutrition in Drosophila: a key role for dFOXO. Physiol. Genomics, published online 7 Nov. 2006 (doi:doi: 10.1152/physiolgenomics.00061.2006).
31. Technikova-Dobrova, Z. et al. Cyclic adenosine monophosphate-dependent phosphorylation of mammalian mitochondrial proteins: enzyme and substrate characterization and functional role. Biochemistry 40, 13941-13947 (2001).
32. Chen, R., Fearnley, I. M., Peak-Chew, S. Y. & Walker, J. E. The phosphorylation of subunits of complex I from bovine heart mitochondria. J. Biol. Chem. 279, 26036-26045 (2004).
33. Pocsfalvi, G. et al. Phosphorylation of B14.5a subunit from bovine heart complex I identified by titanium dioxide selective enrichment and shotgun proteomics. Mol. Cell Proteomics 6, 231-237 (2006).
34. Bellomo, F. et al. Regulation by the cAMP cascade of oxygen free radical balance in mammalian cells. Antioxid. Redox Signal. 8, 495-502 (2006).
35. Papa, S. et al. Complex I and the cAMP cascade in human physiopathology. Biosci. Rep. 22, 3-16 (2002).
36. Piccoli, C. et al. cAMP controls oxygen metabolism in mammalian cells. FEBS Lett. 580, 4539-4543 (2006).
37. Kurtz, A. et al. Somatic mitochondrial DNA mutations in neurofibromatosis type 1-associated tumors. Mol. Cancer Res. 2, 433-441 (2004).
38. Petros, J. A. et al. mtDNA mutations increase tumorigenicity in prostate cancer. Proc. Natl. Acad. Sci. USA 102, 719-724 (2005).
39. Brandon, M., Baldi, P. & Wallace, D. C. Mitochondrial mutations in cancer. Oncogene 25, 4647-4662 (2006).
40. Burdon, R. H. Superoxide and hydrogen peroxide in relation to mammalian cell proliferation. Free Radic. Biol. Med. 18, 775-794 (1995).
41. Barrientos, A. In vivo and in organello assessment of OXPHOS activities. Methods 26, 307-316 (2002).
42. Williams, M. D. et al. Increased oxidative damage is correlated to altered mitochondrial function in heterozygous manganese superoxide dismutase knockout mice. J. Biol. Chem. 273, 28510-28515 (1998).
43. Beauchamp, C. & Fridovich, I. Superoxide dismutase: improved assays and an assay applicable to acrylamide gels. Anal. Biochem. 44, 276-287 (1971).
44. Beers, R. & Sizer, I. A spectrophotometric method for measuring the breakdown of hydrogen peroxide by catalase. J. Biol. Chem. 195, 133-140 (1952).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations, and all methods and systems herein can be used in combination. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method of screening for a modulator of aging or longevity, the method comprising:
providing a non-human animal with an artificial mutation in, or an artificial disruption of expression of, a gene that encodes a component of or that regulates an adenylyl cyclase/cyclic AMP/protein kinase A pathway in the animal, wherein the mutation or disruption is correlated with an aging or longevity trait for the non-human animal;
administering the modulator to the non-human animal; and,
monitoring an effect of the modulator on a phenotype of the non-human animal, wherein the phenotype is correlated to said mutation or disruption.

2. The method of claim 1, wherein the mutation is in a gene selected from the group consisting of: a neurofibromatosis-1 gene, an adenylyl cyclase gene, a cAMP phosphodiesterase gene, and a protein kinase A gene.

3. The method of claim 2, wherein the mutation results in inactivation or overexpression of the neurofibromatosis-1 gene.

4. The method of claim 1, wherein the non-human animal is an insect.

5. (canceled)

6. The method of claim 4, wherein the insect is a Drosophila melanogaster.

7. (canceled)

8. (canceled)

9. The method of claim 1, wherein administering the modulator to the non-human animal comprises feeding the modulator to the non-human animal.

10. The method of claim 1, wherein the phenotype is a life span phenotype and monitoring the effect of the modulator comprises performing a longevity assay that measures the life span of the animal in presence of the modulator.

11. The method of claim 1, wherein the phenotype comprises a stress resistance phenotype and monitoring the effect of the modulator comprises performing a stress resistance assay that measures stress resistance of the animal in presence of the modulator.

12. The method of claim 11, wherein the stress resistance phenotype comprises reduced resistance to heat or oxidative stress as compared to an isogenic or near isogenic animal that lacks the mutation or disruption, and wherein monitoring the effect of the modulator comprises detecting increased resistance to heat or oxidative stress caused by the modulator.

13. The method of claim 1, wherein the phenotype comprises a physical activity or locomotion phenotype and monitoring the effect of the modulator comprises performing a physical activity assay that measures physical activity of the animal in presence of the modulator.

14. The method of claim 13, wherein the animal is an insect and the physical activity assay comprises measuring up climbing/escape response activity of the insect.

15. The method of claim 1, wherein the phenotype comprises an alteration in mitochondrial respiration and monitoring the effect of the modulator comprises performing a mitochondrial respiration activity assay that measures mitochondrial respiration in cells or tissues of the animal, or in an extract thereof, in presence of the modulator.

16. The method of claim 1, wherein the phenotype comprises a mitochondrial respiration trait and monitoring the effect of the modulator comprises performing a mitochondrial respiration activity assay that measures mitochondrial respiration in the animal, in cells or tissues of the animal, or in an extract thereof, after administration of the modulator.

17. The method of claim 1, wherein the phenotype comprises a trait selected from the group consisting of:
(a.) cAMP concentration in the animal, in cells or tissues of the animal, or in an extract thereof,
(b.) complex I activity in the animal, in cells or tissues of the animal, or in an extract thereof,
(c.) citrate synthase activity in the animal, in cells or tissues of the animal, or in an extract thereof,
(d.) mitochondrial ROS production in the animal, in cells or tissues of the animal, or in an extract thereof,
(e.) mitochondrial respiratory control ratio (state III O₂ consumption rate/state IV O₂ consumption rate) in the animal, in cells or tissues of the animal, or in an extract thereof;
(f.) ATP production rate when metabolizing NADH-linked substrates in the animal, in cells or tissues of the animal, or in an extract thereof;
(g.) aconitase activity in the animal, in cells or tissues of the animal, or in an extract thereof,
(h.) superoxide dismutase or catalase activity in the animal, in cells or tissues of the animal, or in an extract thereof; and
(i.) reproductive capacity of the animal.

18. The method of claim 10 wherein the phenotype of the animal in the presence of the modulator is compared to that of an isogenic or nearly isogenic animal in the absence of the modulator.

19. The method of claim 1, wherein the modulator is a cAMP analog, an antioxidant, a catalytic antioxidant, or a metalloporphyrin catalytic antioxidant.

20. The method of claim 1, wherein, following the monitoring step, the modulator is administered to a cell or animal model to test the modulator for anti-cancer or anti-tumor activity.

21. The method of claim 1, wherein, following the monitoring step, the modulator is administered to a patient who has cancer.

22-29. (canceled)

30. A method of treating an NF1 disorder, the method comprising: administering a catalytic antioxidant to a patient suffering from the disorder.

31-45. (canceled)

46. A system for screening for modulator compounds that modulate an aging related behavioral trait, the system comprising:
an array of insects in containers; and,
a behavior monitoring module that monitors physical activity of the insects in the array following administration of the modulator compounds.

47-52. (canceled)
